(19) United States
(12) Patent Application Publication
(10) Pub. No.: US 2019/0194319 A1
(43) Pub. Date: Jun. 27, 2019

(54) ANTI-ASCL1 ANTIBODIES AND METHODS OF USE

(71) Applicant: ABBVIE STEMCENTRX LLC, NORTH CHICAGO, IL (US)

(72) Inventors: JEFFREY BERNSTEIN, SAN FRANCISCO, CA (US); LAURA SAUNDERS, SAN FRANCISCO, CA (US); SHEILA BHEDDAH, SAN FRANCISCO, CA (US); ENYING YOU, SOUTH SAN FRANCISCO, CA (US); CASEY FRANKLIN, PACIFICA, CA (US); TIEN YIN YAU, SAN FRANCISCO, CA (US); GRAYSON KOCHI, SAN MATEO, CA (US)

(73) Assignee: ABBVIE STEMCENTRX LLC, NORTH CHICAGO, IL (US)

(21) Appl. No.: 16/302,995

(22) PCT Filed: May 19, 2017
(86) PCT No.: PCT/US17/33597

§ 371 Date: Nov. 19, 2018

Related U.S. Application Data
(60) Provisional application No. 62/339,757, filed on May 20, 2016.
(60) Provisional application No. 62/505,470, filed on May 12, 2017.

(51) Int. Cl. C07K 16/28; A61K 39/395; A61K 51/10; A61K 49/00; C07K 16/40; G01N 33/563; C07K 16/46

(52) U.S. Cl. CPC C07K 16/28; A61K 39/395; A61K 51/10; C07K 16/46; C07K 16/40; G01N 33/563; A61K 49/0058

(57) ABSTRACT

Compositions comprising Achaete-scute homolog 1 (ASCL1) antibodies, and methods comprising ASCL1 antibody compositions for detecting ASCL1 expression as a diagnostic biomarker, are provided.

### CROSS REFERENCED APPLICATIONS

This application claims the benefit of U.S. Provisional Application No. 62/339,757 filed on May 20, 2016 and U.S. Provisional Application No. 62/505,470 filed on May 12, 2017, each of which is incorporated herein by reference in its entirety.

### SEQUENCE LISTING

This application contains a sequence listing that has been submitted in ASCII format via EFS-Web and is hereby incorporated by reference in its entirety. Said ASCII copy, created on May 16, 2017, is named SC7201WOO1 Sequence_Listing_ST25 and is 53.5 KB (54,812 bytes) in size.

### FIELD OF THE INVENTION

This application generally relates to compositions and methods for detecting, diagnosing or monitoring proliferative disorders such as cancer, including methods for identifying an individual having cancer for tailoring treatment or monitoring progression of a cancer. In a broad aspect, the present invention relates to Achaete-scute homolog 1 (ASCL1) antibodies and antibody compositions and their use for detecting ASCL1 expression as a diagnostic biomarker. The invention further relates to assays, tests, and an article of manufacture, e.g., an assay, test and/or a diagnostic kit, a companion diagnostic assay or a kit comprising the compositions described herein, and a method of manufacturing and using said article.

### BACKGROUND OF THE INVENTION

Proliferative disorders, such as cancer, are among the leading causes of death worldwide.

It is estimated that the number of new cancer cases will rise to twenty two million within the next two decades. Cancer is generally diagnosed based on conventional diagnostic assays and therapeutic options and regimens are typically selected based on determinations made from these assays. There have been some recent advances in characterizing tumors at the molecular level to identify specific biomarkers, such as nucleic acids or proteins associated with specific tumors types or subsets of tumor cells. However, the availability of diagnostic assays based on specific biomarkers is limited, in part due to the paucity of known biomarkers to reliably identify and classify/categorize tumor types and/or stages. Further, proper identification and classification of tumor types and/or stages is critical for tailoring targeted and effective treatment of the tumor. Such informed and effective personalized treatment options can result in improved patient care and enhanced treatment outcomes.

Patients with refractory and metastatic cancer are of particular concern as majority of patients with metastatic cancer eventually run out of treatment options for their tumors. These patients have limited options after their tumor has progressed on standard front line and second line (and sometimes third line and beyond) therapies. Thus, there is a pressing need in the art to develop improved diagnostic tools based on reliable biomarkers, particularly for early detection (before tumor progression), and to develop more targeted, tailored and potent therapies for cancer patients.

### SUMMARY OF THE INVENTION

In certain embodiments, the present invention provides an anti-ASCL1 antibody that comprises or competes for binding to a human ASCL1 protein (SEQ ID NO: 1) with an antibody comprising a light chain variable region set forth as SEQ ID NO: 21 and a heavy chain variable region set forth as SEQ ID NO: 23; a light chain variable region set forth as SEQ ID NO: 25 and a heavy chain variable region set forth as SEQ ID NO: 27; a light chain variable region set forth as SEQ ID NO: 29 and a heavy chain variable region set forth as SEQ ID NO: 31; a light chain variable region set forth as SEQ ID NO: 33 and a heavy chain variable region set forth as SEQ ID NO: 35; a light chain variable region set forth as SEQ ID NO: 37 and a heavy chain variable region set forth as SEQ ID NO: 39; a light chain variable region set forth as SEQ ID NO: 41 and a heavy chain variable region set forth as SEQ ID NO: 43; a light chain variable region set forth as SEQ ID NO: 45 and a heavy chain variable region set forth as SEQ ID NO: 47; a light chain variable region set forth as SEQ ID NO: 49 and a heavy chain variable region set forth as SEQ ID NO: 51; a light chain variable region set forth as SEQ ID NO: 53 and a heavy chain variable region set forth as SEQ ID NO: 55; a light chain variable region set forth as SEQ ID NO: 57 and a heavy chain variable region set forth as SEQ ID NO: 59; a light chain variable region set forth as SEQ ID NO: 61 and a heavy chain variable region set forth as SEQ ID NO: 63; a light chain variable region set forth as SEQ ID NO: 65 and a heavy chain variable region set forth as SEQ ID NO: 67; a light chain variable region set forth as SEQ ID NO: 69 and a heavy chain variable region set forth as SEQ ID NO: 71; or a light chain variable region set forth as SEQ ID NO: 21 and a heavy chain variable region set forth as SEQ ID NO: 73.

In a further aspect, the invention comprises an antibody that binds to human ASCL1 comprising a light chain variable region and a heavy chain variable region, wherein the light chain variable region has three CDRs of a light chain variable region set forth as SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65 or SEQ ID NO: 69 and the heavy chain variable region has three CDRs of a heavy chain variable region set forth as SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO:59 and SEQ ID NO: 63, SEQ ID NO: 67, SEQ ID NO: 71 or SEQ ID NO: 73. As described in some detail below the CDRs may be defined according to Kabat, Chothia, MacCallum or AbM methodology.

In certain embodiments, the anti-ASCL1 antibody of the invention is selected from the group consisting of a monoclonal antibody, primatized antibody, multispecific antibody, bispecific antibody, monovalent antibody, multivalent antibody, anti-idiotypic antibody, diabody, Fab fragment, F(ab′)2 fragment, Fv fragment, and ScFv fragment; or an immunoreactive fragment thereof.

In other embodiments, the anti-ASCL1 antibody of the invention is selected from the group consisting of a chimeric antibody, a CDR-grafted antibody, and a humanized antibody. In certain selected embodiments the anti-ASCL1 antibody is a murine monoclonal antibody.

In another aspect, the invention comprises a method of detecting, diagnosing, or monitoring cancer (e.g. small cell lung cancer or prostate cancer) in a subject, the method comprising the steps of contacting (e.g. in vitro or in vivo) tumor cells with an ASCL1 detection agent and detecting the ASCL1 agent associated with the tumor cells. In selected embodiments the detection agent shall comprise an anti-ASCL1 antibody or a nucleic acid probe that associates with a ASCL1 genotypic determinant. In related embodiments the diagnostic method will comprise immunohistochemistry (IHC) or in situ hybridization (ISH). In other embodiments the method will comprise contacting a circulating tumor cell with an anti-ASCL1 antibody. Those of skill in the art will further appreciate that such ASCL1 detection agents may be labeled or associated with effectors, markers or reporters as disclosed below and detected using any one of a number of standard in vivo imaging techniques (e.g., MRI, CAT scan, PET scan, etc.). In other embodiments the ASCL1 antibody will not be directly labelled and will be detected using a secondary agent that is detectable (e.g., a labelled anti-murine antibody).

In certain embodiments, the tumor sample is chemically fixed or paraffin embedded. In other embodiments, the tumor sample is characterized by a poorly differentiated neuroendocrine phenotype or is at risk of transitioning to a neuroendocrine phenotype. In yet other embodiments, the tumor or tumor sample comprises lung, prostate, breast, ovary, genitourinary tract, gastrointestinal tract, thyroid or kidney cancer. In certain embodiments, the tumor or tumor sample is resistant to other therapies, or is recurrent, or has metastasized.

In certain embodiments, the present invention provides a method for identifying or selecting a subject for undergoing a targeted therapy comprising diagnosing a subject using any of the anti-ASCL1 compositions and detection methods of the invention, and tailoring a targeted therapy based on the outcome. The invention further provides a method for selecting patients having an ASCL1⁺ tumor for administering prophylactic and therapeutic interventions based on one or more downstream targets of ASCL1 (e.g., DLL3), and providing a tailored regimen for preventing or treating a tumor before it progresses to stage where it becomes resistant to a targeted cancer therapy. In certain embodiments, the invention provides a method for targeting an ASCL1 downstream target (e.g., DLL3) with an antibody composition (e.g., an anti-DLL3 antibody or an anti-DLL3 antibody drug conjugate (ADC)) for preventing or treating an adenocarcinoma at risk of transitioning to a neuroendocrine phenotype.

To this end it will be appreciated that certain aspects of the instant invention comprise the use of ASCL1 antibodies for immunohistochemistry to provide a treatment threshold comprising a selected H-score or percentage of positively stained ASCL1 cells. More particularly ASCL1 IHC may be used as a diagnostic tool to aid in the diagnosis or prognosis of various proliferative disorders and to monitor the potential response to treatments including DLL3 ADC therapy. In this respect and as shown in the Examples below immunohistochemistry techniques may be used to derive an H-score as known in the art. Such H-scores (i.e., those 90 and above on a 300 point scale) may be used to indicate which patients may be amenable to treatment with DLL3 ADC compositions as set forth herein as well as being used to guide treatment decisions and determine dosing regimens and timing. In other embodiments the percentage of positively stained ASCL1 cells in the tumor may be used to indicate which patients may be susceptible to treatment with the disclosed DLL3 ADCs.

In other embodiments the invention comprises a method of treating a subject having a tumor comprising tumor cells wherein ≥10% of the tumor cells exhibit 1+ intensity or greater when stained with an ASCL1 antibody and examined in accordance with standard IHC protocols comprising the step of administering an anti-DLL3 ADC.

In a similar vein the present invention also provides kits or devices and associated methods that are useful in the diagnosis, monitoring or treatment of ASCL1 associated disorders such as cancer. To this end the present invention preferably provides an article of manufacture useful for detecting or diagnosing ASCL1 associated disorders comprising a receptacle containing an ASCL1 detecting agent and instructional materials for using said ASCL1 detecting agent to monitor or diagnose the ASCL1 associated disorder (e.g., a DLL3+ cancer) or provide a dosing regimen or treatment schedule for the same. In selected embodiments the devices and associated methods will comprise the step of contacting a tumor, tumor sample or at least one circulating tumor cell. In other embodiments the disclosed kits will comprise instructions, labels, inserts, readers or the like indicating that the kit or device is used for the diagnosis, monitoring or treatment of a ASCL1 associated cancer or provide a dosing regimen for the same.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description.

### BRIEF DESCRIPTION OF THE FIGURES

FIGS. 1A-1F provide annotated amino acid and nucleic acid sequences wherein FIGS. 1A and 1B show contiguous amino acid sequences of the light chain (FIG. 1A) and heavy chain (FIG. 1B) variable regions (SEQ ID NOS: 21-73, odd numbers) of exemplary murine anti-ASCL1 antibodies, FIG. 1C shows nucleic acid sequences encoding the aforementioned light and heavy chain variable regions (SEQ ID NOS: 20-72, even numbers) and FIGS. 1D-1F depict the CDRs of the light and heavy chain variable regions of the SC72.165, SC72.181, and SC72.216 murine antibodies as determined using Kabat, Chothia, ABM and Contact methodology;

FIG. 2 provides, in tabular form, microarray and immunohistochemistry (IHC) results for ASCL1 expression in various lung, colorectal and pancreatic cancer PDX samples where the IHC results are generated using exemplary antibodies of the instant invention; and

FIG. 3 provides, in tabular form, evidence of ASCL1 antibody binding data using various ELISA assays employing samples allowing for intracellular antigen binding (IC ratio), cell lysates (CL ratio) and purified antigens (ELISA positive ratio).

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

### I. Introduction

Human ASCL1 is a 25 kilodalton (kDa) basic helix-loop-helix (bHLH) transcription factor which acts as a master lineage transcription factor orchestrating tissue-specific gene cascade in diverse tissue types. In the context of cancer, human ASCL1 protein was originally discovered in association with neuroendocrine tumors. It has been shown that an elevated expression of ASCL1 characterizes the differentiation of a tumor into a tumor which exhibits neuroendocrine features. ASCL1 is also expressed in tumors exhibiting poor differentiation to a neuroendocrine phenotype, tumors which are undergoing a transition to a neuroendocrine phenotype, tumors which include cells having neuroendocrine features, including tumors which do not exhibit neuroendocrine features.

The incidence of neuroendocrine tumors has markedly increased over the past two decades and is continuing to rise steadily. Various types of neuroendocrine tumors can occur in many different locations in the body, grow differently, and produce different symptoms depending on the location, and whether or not the neuroendocrine tumor is functional or nonfunctional. Functioning neuroendocrine tumors are defined based upon the presence of clinical symptoms due to excess hormone secretion by the tumor. Nonfunctional neuroendocrine tumors do not secrete hormones; however, they may produce symptoms caused by the tumor's growth.

Neuroendocrine tumors often do not cause symptoms early in the disease process, and therefore, they are sometimes misdiagnosed, or diagnosed at a late stage (e.g., after metastasis). Therefore, identifying neuroendocrine tumors early on, including classifying the disease stage is an important step in planning appropriate treatment. The invention provides novel antibodies and their use for detecting ASCL1 expression as a diagnostic biomarker for various tumors, including neuroendocrine tumors.

### II. ASCL1 Physiology

Achaete-scute homolog 1 (ASCL1, also known as Ash1, hASH-1, bHLHa46 or basic-helix-loop-helix protein 46) is a 25 kDa basic helix-loop-helix (bHLH) protein. In humans, the bHLH superfamily comprises over 100 genes which encode transcription factors that bind as dimers with other bHLH proteins at DNA consensus sequences termed E-boxes (5′-CANNTG-3′). As is apparent from their name, bHLH proteins contain two helices, one of which contains basic amino acid residues. Together, the two protein subunits of the dimer comprise a parallel four helix bundle that positions the basic helices to allow interactions between the protein and specific nucleotide residues in the major groove of the DNA at the E-box. Some bHLH proteins, such as E12 and E47, are expressed in many tissues, and may form homodimers to control gene expression. Other bHLH proteins, such as ASCL1, show a more tissue restricted expression pattern and may or not be capable of acting as homodimers; instead, they typically heterodimerize with class I proteins, thereby conveying tissue-specific gene expression patterns in their response genes. Cell-type specific bHLH proteins are involved in numerous biological processes, including but not limited to, neurogenesis, cardiogenesis, myogenesis and hematopoiesis (PMID: 20219281).

Representative ASCL1 protein orthologs include, but are not limited to, human (NP_004307; SEQ ID NO: 1), chimpanzee (XP_009424458), cynomolgus monkey (XP_005572101), rat (NP_032579) and mouse (NP_032579). In humans, the ASCL1 gene consists of 2 exons spanning approximately 3 kBp at chromosome 12q23.2. Transcription of the human ASCL1 locus yields a 2490 nucleotide transcript (NM_004316) that encodes a 236 amino acid protein (NP_004307). No alternative spliced transcripts or variant proteins have been reported. The amino acid sequence of human ASCL1 (SEQ ID NO: 1) is shown immediately below.

NP_004307.2_achaete-scute homolog 1 [Homo sapiens]
MESSAKMESGGAGQQPQPQPQQPFLPPAACFFATAAAAAAAAAAAAAQSA
QQQQQQQQQQQQAPQLRPAADGQPSGGGHKSAPKQVKRQRSSSPELMRCK
RRLNFSGFGYSLPQQQPAAVARRNERERNRVKLVNLGFATLREHVPNGAA
NKKMSKVETLRSAVEYIRALQQLLDEHDAVSAAFQAGVLSPTISPNYSND
LNSMAGSPVSSYSSDEGSYDPLSPEEQELLDFTNWF

Human ASCL1 was first identified in 1993 as a transcription factor highly expressed in neuroendocrine tumors, including small cell lung cancer (SCLC) and medullary thyroid cancer (PMID: 8390674). Since then, studies into the function of ASCL1 have revealed its role in myogenesis (e.g., PMID: 8662987) and neurogenesis (reviewed in PMID: 25520623) or as a proneuroendocrine transcription factor required for development of lung neuroendocrine cells (PMID: 9126746), thyroid parafollicular cells (PMID: 17103415), chromaffin cells of the adrenal medulla (PMID: 12361965), and glandular stomach neuroendocrine cells (PMID: 18173746). In each of these tissues, ASCL1 acts as a master lineage transcription factor orchestrating tissue-specific gene cascades.

With regard to its regulatory role in neurogenesis, Ascl1 was found to be one of three transcription factors that when expressed together could reprogram mouse embryonic and postnatal fibroblasts into functional neurons in vivo (PMID: 20107439). These results were recapitulated in human fibroblasts (PMID: 21617644), and together indicate that cells, or at least certain cell types, can have considerable plasticity in their fates. More recent studies have revealed that the bHLH transcription factors Ascl1, Hes1 and Olig2 are expressed in neural progenitor cells (NPCs) in an oscillatory manner, due to complex feedback loops at both the RNA and protein levels (PMID: 24179156). The multipotent and proliferative state of the NPCs were correlated with the oscillatory expression of these factors, whereas a differentiated state correlated with sustained expression of a single factor—Ascl1 with neurons, Hes1 with astrocytes and Olig2 with oligodendrocytes. Interestingly, the oscillations of Hes1 and Ascl1 were out-of-phase with one another, and inactivation of Hes1 abolished the Ascl1 oscillation, consistent with previous reports of a direct repression of ASCL1 by the Notch effector gene HES1 in SCLC lines (PMID: 9144241; PMID: 11940670). The relatively short oscillation period (2-3 hrs) for the proteins may give rise to a characteristic “salt and pepper” expression pattern of one or the other in a field of cells, indicative of a snapshot of cells at a specific timepoint rather than indications of a commitment to differentiation (PMID: 25520623). In contrast to these oscillations, sustained Ascl1 expression in NPCs during G1 occurred just before asymmetric division of the NPC into a daughter NPC, which resumed Ascl1 and Hes1 oscillations, and an Ascl1-expressing cell committed to neuronal differentiation. What determines the transition to sustained expression remains unclear, although changes in the period and amplitude of the oscillations may be related to fluctuations in the amounts of Notch intracellular domain. It has been proposed that a similar oscillatory behavior of Ascl I and other bHLH factors can explain the ultimate expression of the individual factors in discrete, cross-repressive domains of the developing spinal cord (PMID: 24257627; PMID: 15901662). Taken together these studies imply that ASCL1 can support both stem-ness and proliferation, or cell-fate determination, depending upon context, duration of expression and the absolute expression level.

In the context of cancer, the human ASCL1 protein was discovered in association with neuroendocrine tumors. The linkage between the expression of ASCL1 and the neuroendocrine phenotype in tumors was demonstrated using antisense oligonucleotides targeted to ASCL1, which depleted ASCL1 expression in SCLC lines, leading to concomitant reduction in expression of neuroendocrine markers (PMID: 9126746). Other studies have shown that expression of ASCL1 is linked to survival of SCLC (PMID: 16322211; PMID: 19176379) and neuroendocrine non-SCLC (PMID: 25267614). Therefore ASCL1 can be considered a lineage addiction oncogene, which in a normal developmental context promotes neural or neuroendocrine differentiation (most likely from a multipotent precursor cell) yet when it is reactivated or dysregulated outside of normal development processes, is responsible for a phenotypic plasticity, commitment to a partially differentiated or partial multipotential state, and/or cancerous cell phenotype with strong neuroendocrine features.

Interestingly, it has been shown that ASCL1 can bind the promoter of the Notch signaling pathway protein DLL3, either as a homodimer or as a dimer with the bHLH protein NEUROG2 (PMID: 19389376). Thus, while the Notch signaling effector protein HES1 can suppress ASCL1, this ASCL1/DLL3 interaction suggests that ASCL1 itself may induce expression of a negative regulator of Notch signaling. It has also been shown that siRNA-mediated knock-down of ASCL1 leads to concomitant reductions in DLL3 mRNA levels (PMID: 19176379). DLL3 is known to be overexpressed in many neuroendocrine tumors (U59089615, US9089616) and has been demonstrated to be a tractable target for development of antibody-drug conjugates for the treatment of high-grade pulmonary neuroendocrine tumors (PMID: 26311731; US9107961). Therefore, ASCL1 expression may be a useful biomarker for neuroendocrine tumors.

### III. Antibodies

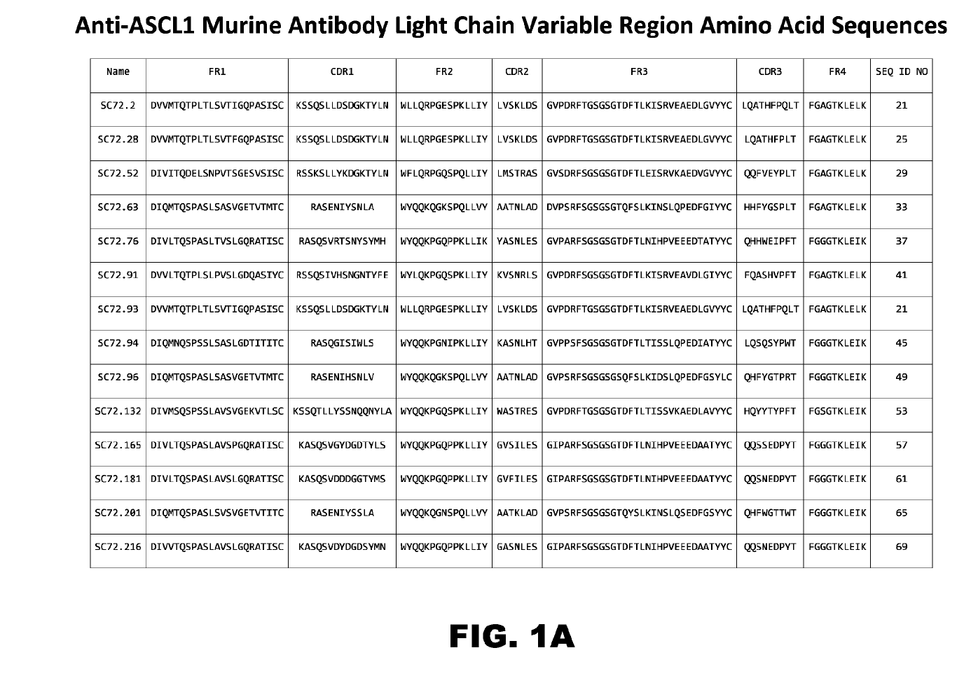

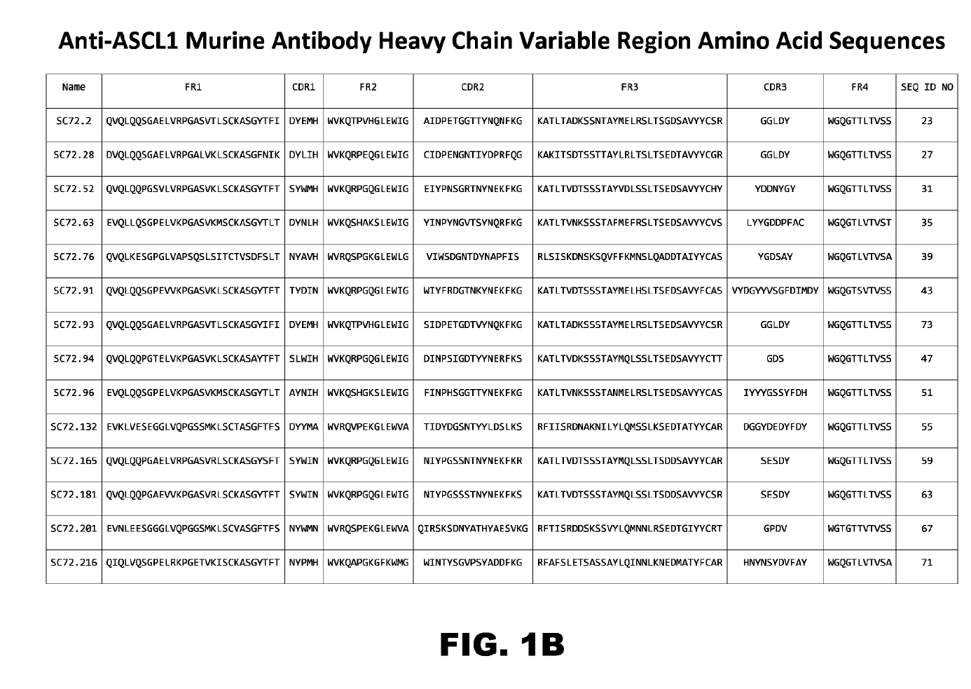

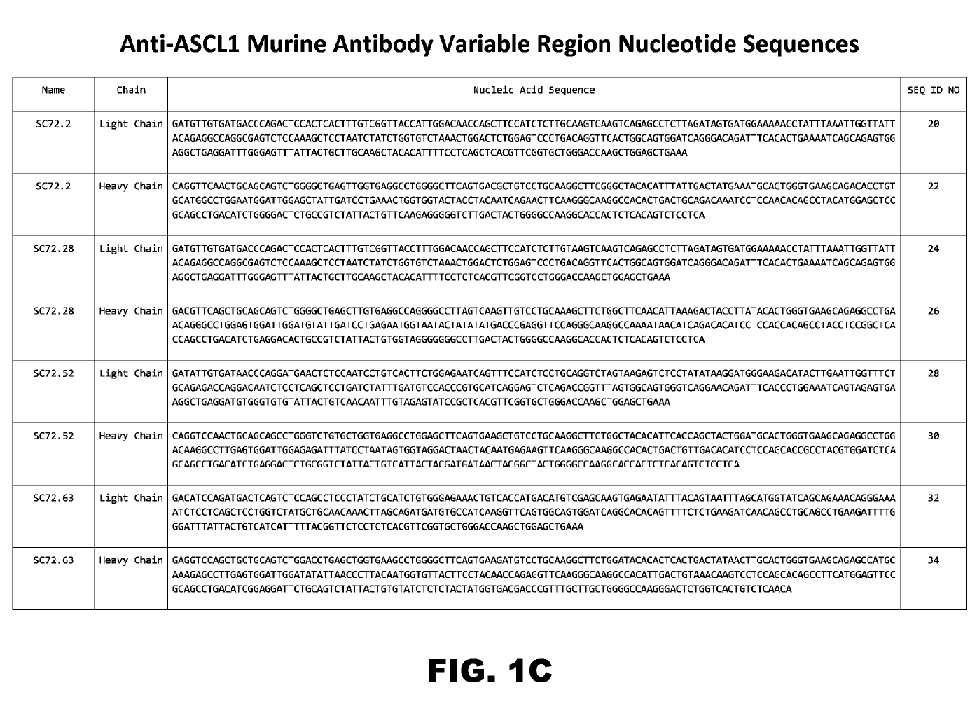

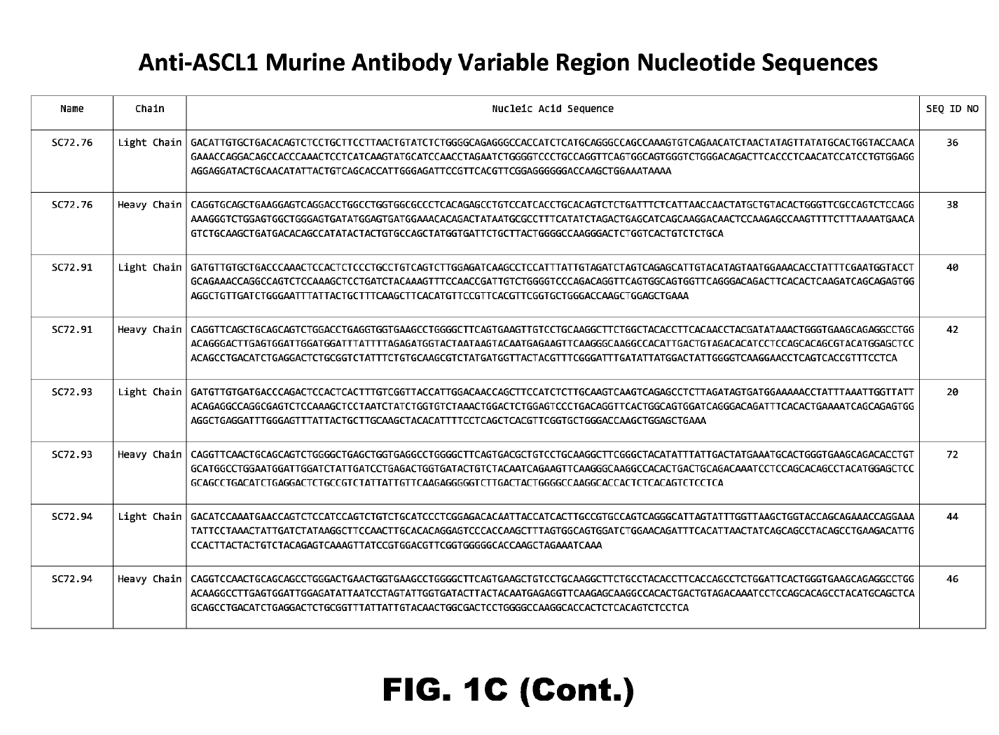

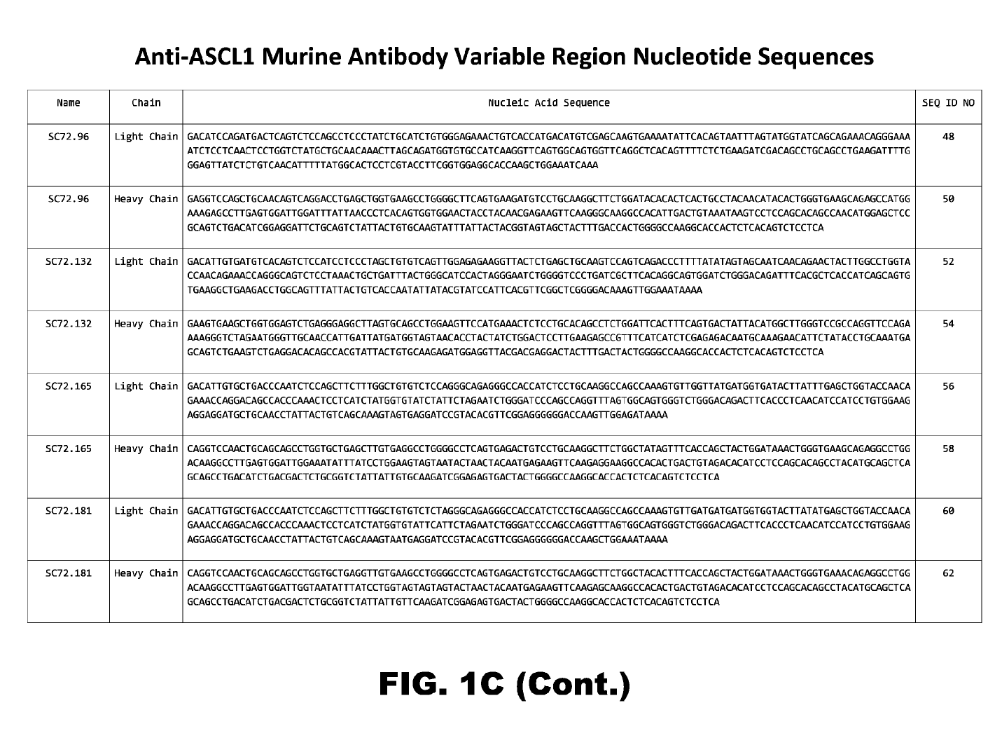

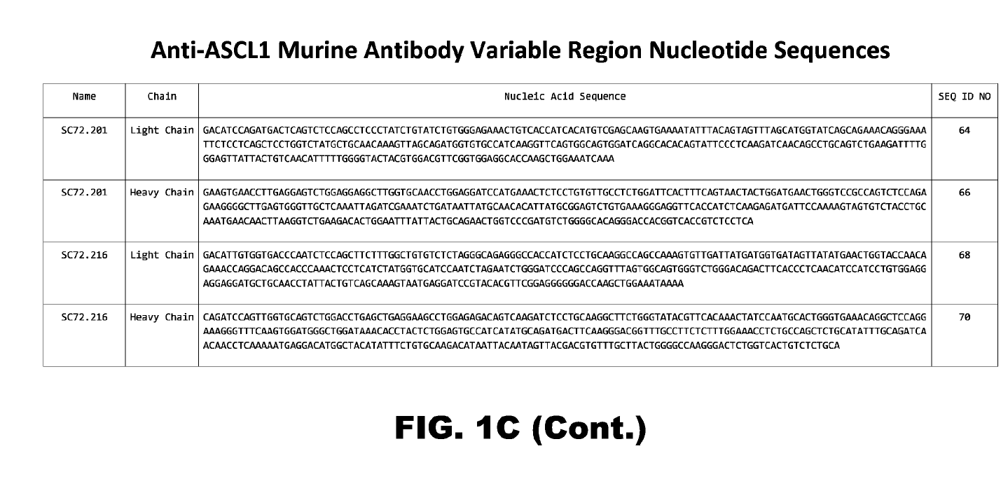

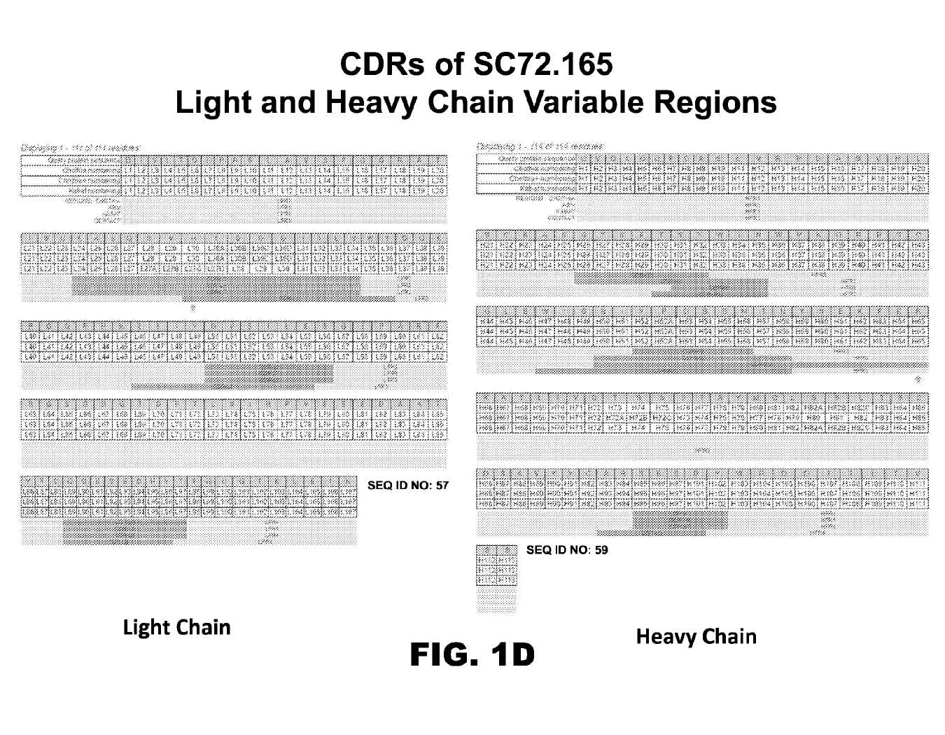

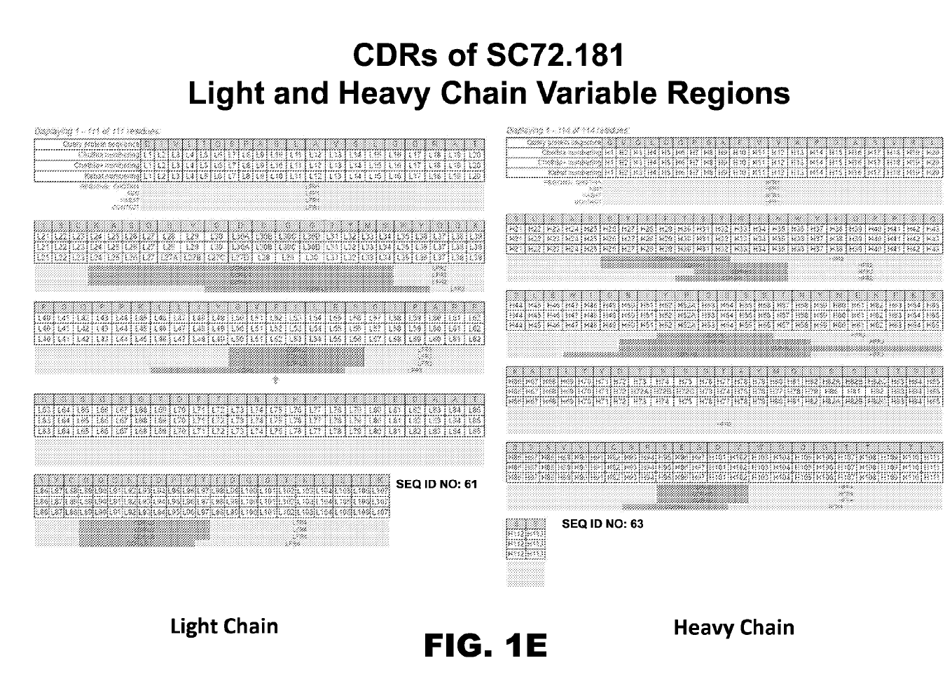

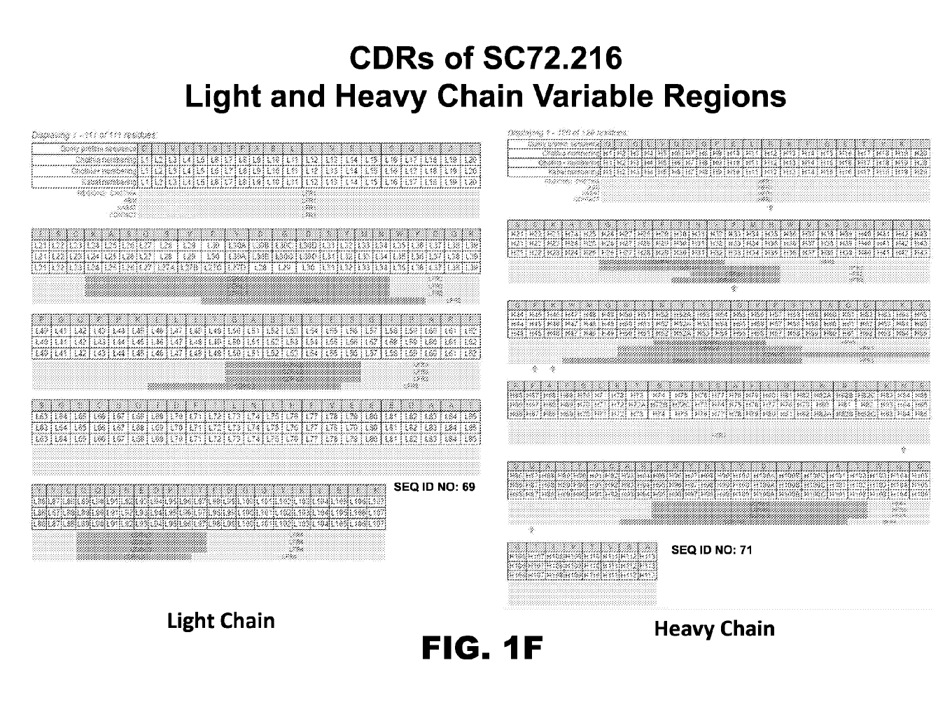

The invention provides novel anti-ASCL1 antibodies and compositions comprising the novel anti-ASCL1 antibodies. The anti-ASCL1 antibodies of the invention were produced as described in the Examples provided herein. The ASCL1 antibodies of the invention can be used in any of the methods provided herein. FIGS. 1A-B provide the annotated amino acid sequences of exemplary murine anti-ASCL1 binding or targeting domains, termed SC72.2, SC72.28, SC72.52, SC72.63, SC72.76, SC72.91, SC72.94, SC72.96, SC72.132, SC72.165, SC72.181, SC72.201, SC72.216 and SC72.93. The amino acid sequences of the light chain variable regions and heavy chain variable regions of the ASCL1 antibodies are also depicted in FIGS. 1A and 1B, respectively, and are represented by SEQ ID NOS: 21-73, odd numbers. The nucleotide sequences of the light chain variable regions and heavy chain variable regions of the ASCL1 antibodies are depicted in FIG. 1C, and are represented by SEQ ID NOS: 20-72, even numbers.

A. Antibody Structure

Antibodies and variants and derivatives thereof, including accepted nomenclature and numbering systems, have been extensively described, for example, in Abbas et al. (2010), Cellular and Molecular Immunology (6^{th} Ed.), W. B. Saunders Company; or Murphey et al. (2011), Janeway's Immunobiology (8^{th} Ed.), Garland Science.

An “antibody” or “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies, recombinantly produced antibodies, intrabodies, multi specific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ, based on the amino acid sequences of their constant domains.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of the antibody. ScFv fragments (for single chain fragment variable), which can be obtained by genetic engineering, associates in a single polypeptide chain, the VH and the VL region of an antibody, separated by a peptide linker.

As used herein, the assignment of amino acids to each domain, framework region and CDR may be in accordance with one of the schemes provided by Kabat et al. (1991) Sequences of Proteins of Immunological Interest (5^{th} Ed.), US Dept. of Health and Human Services, PHS, NIH, NIH Publication no. 91-3242; Chothia et al, 1987, PMID: 3681981; Chothia et al, 1989, PMID: 2687698; MacCallum et al.,1996, PMID: 8876650; or Dubel, Ed. (2007) Handbook of Therapeutic Antibodies, 3^{rd} Ed., Wily-VCII Verlag GmbH and Co or AbM (Oxford Molecular/MSI Pharmacopia) unless otherwise noted. As is well known in the art variable region residue numbering is typically as set forth in Chothia or Kabat. Amino acid residues which comprise CDRs as defined by Kabat, Chothia, MacCallum (also known as Contact) and AbM as obtained from the Abysis website database (infra.) are set out below. Note that MacCallum uses the Chothia numbering system.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat nomenclature system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinforg.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005).

Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. FIGS. 1D-1F appended hereto show the results of such analysis in the annotation of exemplary heavy and light chain variable regions (VH and VL) for the SC72.165, SC72.181 and SC72.216 antibodies. Unless otherwise indicated, all CDRs set forth herein are derived according to the Abysis database website as per Kabat et al.

Murine heavy chain constant region amino acid positions discussed in the invention are well known in the art and may be readily derived, altered or manipulated using conventional genetic engineering and biochemical techniques. In analyzing such sequences numbering systems analogous to the Eu index first described in Edelman et al., 1969, Proc. Natl. Acad. Sci. USA 63(1): 78-85 (describing the amino acid sequence of the myeloma protein Eu, which reportedly was the first human IgG1 sequenced) may be employed. The Eu index of Edelman is also set forth in Kabat et al., 1991 (supra.). Similarly, a numbering system used for human light chain constant region amino acid sequences is set forth in Kabat et al., (supra.) and analogous systems may be used for the murine light chains disclosed herein.

By way of example murine kappa (SEQ ID NO: 2) and lambda (SEQ ID NO: 3) light chain constant region amino acid sequences compatible with the present invention are set forth immediately below:

(SEQ ID NO: 2)
RADAAPTVSIFPPSSEQLTSGGASVVCFLNNFYPKDINVKWKIDGSERQN
GVLNSWTDQDSKDSTYSMSSTLTLTKDEYERHNSYTCEATHKTSTSPIVK
SFNRNEC -
(SEQ ID NO: 3)
GQPKSSPSVTLFPPSSEELETNKATLVCTITDFYPGVVTVDWKVDGTPVT
QGMETTQPSKQSNNKYMASSYLTLTARAWERHSSYSCQVTHEGHTVEKSL
SRADCS -

Similarly, an exemplary IgG1 murine heavy chain constant region amino acid sequence (SEQ ID NO: 4) compatible with the present invention is set forth immediately below:

SEQ ID NO: 4)
AKTTPPSVYPLAPGSAAQTNSMVTLGCLVKGYFPEPVTVTWNSGSLSSGV
HTFPAVLQSDLYTLSSSVTVPSSPRPSETVTCNVAHPASSTKVDKKIVPR
DCGCKPCICTVPEVSSVFIFPPKPKDVLTITLTPKVTCVVVDISKDDPEV
QFSWFVDDVEVHTAQTQPREEQFNSTFRSVSELPIMHQDWLNGKEFKCRV
NSAAFPAPIEKTISKTKGRPKAPQVYTIPPPKEQMAKDKVSLTCMITDFF
PEDITVEWQWNGQPAENYKNTQPIMNTNGSYFVYSKLNVQKSNWEAGNTF
TCSVLHEGLHNHHTEKSLSHSPGK -

The disclosed constant region sequences, or variations or derivatives thereof, may be operably associated with the disclosed heavy and light chain variable regions using standard molecular biology techniques to provide full-length antibodies that may be used as such or incorporated in the diagnostic ADCs of the invention.

Those of skill in the art will appreciate that there are two types of disulfide bridges or bonds in immunoglobulin molecules: interchain and intrachain disulfide bonds. As is well known in the art the location and number of interchain disulfide bonds vary according to the immunoglobulin class and species. While the invention is not limited to any particular class or subclass of antibody, the IgG1 immunoglobulin shall be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for chain association. The IgG1 hinge region contain the cysteines in the heavy chain that form the interchain disulfide bonds, which provide structural support along with the flexibility that facilitates Fab movement.

B. Antibody Generation and Production

Antibodies of the invention can be produced using a variety of methods known in the art.

1. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

In this regard antibodies of the invention may be generated from any ASCL1 determinant that induces an immune response in an immunocompetent animal. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells that may express DLL3 and may comprise a ASCL1 protein, or any of its splice variants, isoforms, homologs or family members, or specific domains, regions or epitopes thereof. An “antigen”, “immunogenic determinant”, “antigenic determinant” or “immunogen” means any ASCL1 protein or any fragment, region or domain thereof that can stimulate an immune response when introduced into an immunocompetent animal and is recognized by the antibodies produced by the immune response. The presence or absence of the ASCL1 (and/or DLL3) determinants contemplated herein may be used to identify a cell, cell subpopulation or tissue (e.g., tumors, tumorigenic cells or CSCs).

Any form of antigen, or cells or preparations containing the antigen, can be used to generate an antibody that is specific for the ASCL1 determinant. As alluded to the term “antigen” is used in a broad sense and may comprise any immunogenic fragment or determinant of the selected target including a single epitope, multiple epitopes, single or multiple domains or the entire extracellular domain (ECD) or protein. The antigen may be an isolated full-length protein, a cell surface protein (e.g., immunizing with cells expressing at least a portion of the antigen on their surface), or a soluble protein (e.g., immunizing with only the ECD portion of the protein) or protein construct (e.g., Fc-antigen). The antigen may be produced in a genetically modified cell. Any of the aforementioned antigens may be used alone or in combination with one or more immunogenicity enhancing adjuvants known in the art. DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

2. Monoclonal Antibodies

In selected embodiments, the invention contemplates use of monoclonal antibodies. As known in the art, the term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following production of multiple monoclonal antibodies that bind specifically to a determinant, particularly effective antibodies may be selected through various screening processes, based on, for example, its affinity for the determinant or rate of internalization. Antibodies produced as described herein may be used as “source” antibodies and further modified to, for example, improve affinity for the target, improve its production in cell culture, reduce immunogenicity in vivo, create multispecific constructs, etc. A more detailed description of monoclonal antibody production and screening is set out below and in the appended Examples.

3. Derived Antibodies

Once source antibodies have been generated, selected and isolated as described above they may be further altered to provide anti-ASCL1 antibodies having improved pharmaceutical characteristics. Preferably the source antibodies are modified or altered using known molecular engineering techniques to provide derived antibodies having the desired therapeutic properties.

Selected embodiments of the invention comprise murine monoclonal antibodies that immunospecifically bind to ASCL1 and that can be considered “source” antibodies. In selected embodiments, antibodies of the invention can be derived from such “source” antibodies through optional modification of the constant region and/or the epitope-binding amino acid sequences of the source antibody. In certain embodiments an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor antibody sequence to provide the derivative antibody (e.g. chimeric or humanized antibodies). These “derived” antibodies can be generated using standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567; Morrison et al., 1984, PMID: 6436822). In some embodiments chimeric antibodies of the instant invention may comprise all or most of the selected murine heavy and light chain variable regions operably linked to human light and heavy chain constant regions. In other selected embodiments, anti-ASCL1 antibodies may be “derived” from the mouse antibodies disclosed herein.

In other embodiments, chimeric antibodies of the invention are “CDR-grafted” antibodies, where the CDRs (as defined using Kabat, Chothia, McCallum, etc.) are derived from a particular species or belonging to a particular antibody class or subclass, while the remainder of the antibody is largely derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In one embodiment the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (donor or source antibody). In certain embodiments, “back mutations” can be introduced into the humanized antibody, in which residues in one or more FRs of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention comprising murine components from source antibodies and human components from acceptor antibodies are provided as set forth in the Examples below.

Various art-recognized techniques can be used to determine which human sequences to use as acceptor antibodies to provide humanized constructs in accordance with the instant invention. Compilations of compatible human germline sequences and methods of determining their suitability as acceptor sequences are disclosed, for example, in Dubel and Reichert (Eds.) (2014) Handbook of Therapeutic Antibodies, 2′ Edition, Wiley-Blackwell GmbH; Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638). The V-BASE directory (VBASE2-Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, (PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

It will be appreciated that the annotated CDRs and framework sequences as provided in the appended FIGS. 1A and 1B are defined as per Kabat et al. using a proprietary Abysis database. However, as discussed herein and shown in FIGS. 1D-1F, one skilled in the art could readily identify CDRs in accordance with definitions provided by Chothia et al., ABM or MacCallum et al. as well as Kabat et al. As such, anti-ASCL1 humanized antibodies comprising one or more CDRs derived according to any of the aforementioned systems are explicitly held to be within the scope of the instant invention.

4. Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation and/or disulfide bonds and modified binding specificity.

Compounds with improved Fc effector functions can be generated, for example, through changes in amino acid residues involved in the interaction between the Fc domain and an Fc receptor (e.g., FcγRI, FcγRIIA and B, F65 RIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas etal., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001). Surprisingly, certain ADCs of the instant invention exhibit protracted terminal half-lives (e.g., on the order of two weeks) without any antibody constant region modifications other than those used to provide optional site-specific conjugates.

5. Fragments

Regardless of which form of antibody (e.g. chimeric, humanized, etc.) is selected to practice the invention it will be appreciated that immunoreactive fragments, either by themselves or as part of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary site-specific fragments include: variable light chain fragments (VL), an variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines as described herein.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

6. Recombinant Production of Antibodies

Antibodies and fragments thereof may be produced or modified using genetic material obtained from antibody producing cells and recombinant technology (see, for example; Dubel and Reichert (Eds.) (2014) Handbook of Therapeutic Antibodies, 2^{nd} Edition, Wiley-Blackwell GmbH; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsC1 banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In selected embodiments the nucleic acid is a cDNA molecule.

Nucleic acids of the invention can be obtained using standard molecular biology techniques. For antibodies expressed by hybridomas (e.g., hybridomas prepared as described in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein or protein fragment, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding murine or human heavy chain constant regions (CH1, CH2 and CH3 in the case of IgG1). The sequences of human and murine heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 4. For a Fab fragment heavy chain gene, the VH-encoding DNA can be operatively linked to another DNA molecule encoding only the heavy chain CH1 constant region.

Isolated DNA encoding the VL region can be converted to a full-length light chain gene (as well as a Fab light chain gene) by operatively linking the VL-encoding DNA to another DNA molecule encoding the light chain constant region, CL. The sequences of murine and human light chain constant region genes are known in the art and DNA fragments encompassing these regions can be obtained by standard PCR amplification. The light chain constant region can be a kappa or lambda constant region, but most preferably is a kappa constant region. Exemplary compatible kappa and lambda light chain constant regions are set forth in SEQ ID NOS: 2 and 3.

Contemplated herein are certain polypeptides (e.g. antigens or antibodies) that exhibit “sequence identity”, sequence similarity” or “sequence homology” to the polypeptides of the invention. For example, a derived humanized antibody VH or VL domain may exhibit a sequence similarity with the source (e.g., murine) or acceptor (e.g., human) VH or VL domain. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. AppL Biosci.,4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,) XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any type of cellular system that can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity). For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under selected conditions. The GS system is discussed in whole or part in connection with EP 0 216 846, EP 0 256 055, EP 0 323 997 and EP 0 338 841 and U.S. Pat. Nos 5,591,639 and 5,879,936. Another compatible expression system for the development of stable cell lines is the Freedom™ CHO-S Kit (Life Technologies).

Once an antibody of the invention has been produced by recombinant expression or any other of the disclosed techniques, it may be purified or isolated by methods known in the art in that it is identified and separated and/or recovered from its natural environment and separated from contaminants that would interfere with diagnostic or therapeutic uses for the antibody or related ADC. Isolated antibodies include antibodies in situ within recombinant cells.

These isolated preparations may be purified using various art-recognized techniques, such as, for example, ion exchange and size exclusion chromatography, dialysis, diafiltration, and affinity chromatography, particularly Protein A or Protein G affinity chromatography. Compatible methods are discussed more fully in the Examples below.

7. Post-Production Selection

No matter how obtained, antibody-producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et at, 1998, PMID: 9600934; Boder et al., 1997, PMID: 9181578; Pepper et al, 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

8. Characteristics of Antibodies

In certain embodiments, antibody-producing cells (e.g., hybridomas or yeast colonies) may be selected, cloned and further screened for favorable properties including, for example, robust growth, high antibody production and, stability, reduced immunogenicity. In other cases characteristics of the antibody may be imparted by selecting a particular antigen (e.g., a specific ASCL1 isoform) or immunoreactive fragment of the target antigen for inoculation of the animal.

In still other embodiments the selected antibodies may be engineered as described above to enhance or refine immunochemical characteristics such as affinity or pharmacokinetics.

8.1. Binding Affinity

Disclosed herein are antibodies that have a high binding affinity for ASCL1. The term “K_{D}” refers to the dissociation constant or apparent affinity of a particular antibody-antigen interaction. An antibody of the invention can immunospecifically bind its target antigen when the dissociation constant K_{D} (k_{off}/kₒₙ) is ≤10⁻⁷M. The antibody specifically binds antigen with high affinity when the K_{D} is ≤5×10⁻⁹ M, and with very high affinity when the K_{D} is ≤b 5×10⁻¹⁰ M. In one embodiment of the invention, the antibody has a K_{D} of ≤10⁻⁹M and an off-rate of about 1×10⁻⁴ /sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}<2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹²M, less than 5×10⁻¹² M, less than 10⁻¹³ M, less than 5×10⁻¹³ M, less than 10⁻¹⁴ M, less than 5×10⁻¹⁴ M, less than 10⁻⁻¹⁵ M or less than 5×10⁻¹⁵ M.

In certain embodiments, an antibody of the invention that immunospecifically binds to ASCL1 may have an association rate constant or kₒₙ (or kₐ) rate (antibody+antigen (Ag)^{k}ₒₙ←antibody−Ag) of at least 10⁵ M⁻¹s⁻¹, at least 2×10⁵ M⁻¹s⁻¹, at least 5×10⁵ M⁻¹s⁻¹, at least 10⁶ M⁻¹s⁻¹ at least 5×10⁶ M⁻¹s⁻¹ at least 10⁷ M⁻¹s⁻¹, at least 5×10⁷ M⁻¹s⁻¹, or at least 10⁸ M⁻¹s⁻¹.

In another embodiment, an antibody of the invention that immunospecifically binds to ASCL1 may have a disassociation rate constant or k_{off} (or k_{d}) rate (antibody+antigen (Ag)^{k}_{off}←antibody−Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻⁻³ s⁻¹, less than 10⁻⁴ s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹ or less than 10⁻¹⁰ s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

8.2. Binning and Epitope Mapping

Antibodies disclosed herein may be characterized in terms of the discrete epitope with which they associate. An “epitope” is the portion(s) of a determinant to which the antibody or immunoreactive fragment specifically binds. Immunospecific binding can be confirmed and defined based on binding affinity, as described above, or by the preferential recognition by the antibody of its target antigen in a complex mixture of proteins and/or macromolecules (e.g. in competition assays). A “linear epitope”, is formed by contiguous amino acids in the antigen that allow for immunospecific binding of the antibody. The ability to preferentially bind linear epitopes is typically maintained even when the antigen is denatured. Conversely, a “conformational epitope”, usually comprises non-contiguous amino acids in the antigen's amino acid sequence but, in the context of the antigen's secondary, tertiary or quaternary structure, are sufficiently proximate to be bound concomitantly by a single antibody. When antigens with conformational epitopes are denatured, the antibody will typically no longer recognize the antigen. An epitope (contiguous or non-contiguous) typically includes at least 3, and more usually, at least 5 or 8-10 or 12-20 amino acids in a unique spatial conformation.

It is also possible to characterize the antibodies of the invention in terms of the group or “bin” to which they belong. “Binning” refers to the use of competitive antibody binding assays to identify pairs of antibodies that are incapable of binding an immunogenic determinant simultaneously, thereby identifying antibodies that “compete” for binding. Competing antibodies may be determined by an assay in which the antibody or immunologically functional fragment being tested prevents or inhibits specific binding of a reference antibody to a common antigen. Typically, such an assay involves the use of purified antigen (e.g., DLL3, ASCL1, or a domain or fragment thereof) bound to a solid surface or cells, an unlabeled test antibody and a labeled reference antibody. Competitive inhibition is measured by determining the amount of label bound to the solid surface or cells in the presence of the test antibody. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more. Conversely, when the reference antibody is bound it will preferably inhibit binding of a subsequently added test antibody (i.e., a DLL3 antibody or ASCL1 antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

Similarly binning techniques comprising surface plasmon resonance are compatible with the instant invention. As used herein “surface plasmon resonance,” refers to an optical phenomenon that allows for the analysis of real-time specific interactions by detection of alterations in protein concentrations within a biosensor matrix. Using commercially available equipment such as the BIAcore™ 2000 system it may readily be determined if selected antibodies compete with each other for binding to a defined antigen.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® ctet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which that group of antibodies binds. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds.

In certain embodiments fine epitope mapping can be performed using phage or yeast display. Other compatible epitope mapping techniques include alanine scanning mutants, peptide blots (Reineke, 2004, PMID: 14970513), or peptide cleavage analysis. In addition, methods such as epitope excision, epitope extraction and chemical modification of antigens can be employed (Tomer, 2000, PMID: 10752610) using enzymes such as proteolytic enzymes (e.g., trypsin, endoproteinase Glu-C, endoproteinase Asp-N, chymotrypsin, etc.); chemical agents such as succinimidyl esters and their derivatives, primary amine-containing compounds, hydrazines and carbohydrazines, free amino acids, etc. In another embodiment Modification-Assisted Profiling, also known as Antigen Structure-based Antibody Profiling (ASAP) can be used to categorize large numbers of monoclonal antibodies directed against the same antigen according to the similarities of the binding profile of each antibody to chemically or enzymatically modified antigen surfaces (U.S.P.N. 2004/0101920).

Once a desired epitope on an antigen is determined, it is possible to generate additional antibodies to that epitope, e.g., by immunizing with a peptide comprising the selected epitope using techniques described herein.

### IV. Diagnostics

The invention provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with a detection agent (e.g., an antibody or nucleic acid probe) capable of specifically recognizing and associating with a ASCL1 determinant and detecting the presence or absence, or level of association of the detection agent in the sample. In selected embodiments the detection agent will comprise an antibody associated with a detectable label or reporter molecule as described herein. In certain other embodiments the ASCL1 antibody will be administered and detected using a secondary labelled antibody (e.g., an anti-murine antibody). In yet other embodiments (e.g., In situ hybridization or ISH) a nucleic acid probe that reacts with a genomic ASCL1 determinant will be used in the detection, diagnosis or monitoring of the proliferative disorder. The anti-ASCL1 antibodies of the invention are further useful in the prognosis, diagnosis, and theragnosis of tumors (e.g., adenocarcinoma at risk of transitioning to a neuroendocrine phenotype).

The invention further provides a method for selecting patients having an ASCL1⁺ tumor for administering prophylactic and therapeutic interventions based on one or more downstream targets of ASCL1 (e.g., DLL3), and providing a tailored regimen for preventing or treating a tumor before it progresses to stage where it becomes resistant to a targeted cancer therapy. In certain embodiments, the invention provides a method for targeting an ASCL1 downstream target (e.g., DLL3) with an antibody composition (e.g., an anti-DLL3 antibody or an anti-DLL3 antibody drug conjugate (ADC)) for preventing or treating an adenocarcinoma at risk of transitioning to a neuroendocrine phenotype.

A. Diagnostic Methods

The anti-ASCL1 antibody compositions of the present invention are useful in in vitro, ex vivo, and in vivo methods for detecting, diagnosing or monitoring proliferative disorders and methods of screening cells from a patient to identify tumor cells including tumorigenic cells. Such methods include identifying an individual having cancer for treatment or monitoring progression or morphological transformation of a cancer. Representative methods include the steps of (a) contacting an anti-ASCL1 antibody with a tumor sample obtained from a subject; and (b) detecting ASCL1 antibodies bound to the tumor sample. Antibodies useful in the invention include those depicted in FIGS. 1A-1B, and variants of such antibodies as described herein above. The ASCL1 antibodies may be detected in the tumor sample by virtue of a detectable label bound to the antibody, or by use of secondary and/or tertiary antibodies to amplify the signal, as is well known in the art.

More generally the presence and/or level(s) of ASCL1 determinant(s) may be measured using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), Polymerase Chain Reaction (PCR, RT-PCR; RT-qPCR) technology, branched oligonucleotide technology, Northern blot technology, oligonucleotide hybridization technology and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In some embodiments, the association of the detection agent with particular cells or cellular components in the sample indicates that the sample may contain tumorigenic cells, thereby denoting that the individual having cancer may be effectively treated with an antibody or ADC as described herein.

In certain preferred embodiments the assays may comprise immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.).

In this regard certain aspects of the instant invention comprise the use of labeled ASCL1 for immunohistochemistry (IHC). More particularly ASCL1 IHC may be used as a diagnostic tool to aid in the diagnosis of various proliferative diseases and to monitor the potential response to treatments, e.g., DLL3 antibody therapy. Compatible diagnostic assays may be performed on tissues that have been chemically fixed (compatible techniques include, but are not limited to: formaldehyde, glutaraldehyde, osmium tetroxide, potassium dichromate, acetic acid, alcohols, zinc salts, mercuric chloride, chromium tetroxide and picric acid) and embedded (compatible methods include but are not limited to: glycol methacrylate, paraffin and resins) or preserved via freezing. Such assays can be used to guide treatment decisions and determine dosing regimens and timing.

Immunohistochemistry techniques may be used to derive an H-score as known in the art using the disclosed ASCL1 antibodies. Briefly tumor sections are viewed (preferably by brightfield microscopy) and ASCL1 expression on sectioned tumor is noted to derive an H-score. The H-score is obtained by the formula: 3×percentage of strongly staining nucleus+2×percentage of moderately staining nucleus+percentage of weakly staining nucleus, giving a range of 0 to 300.

Such ASCL1 H-scores may be used to indicate which patients may be amenable to treatment with a suitable composition (e.g., an anti-DLL3 ADC). ASCL1 H-scores of approximately 90, approximately 100, approximately 110, approximately 120, approximately 130, approximately 140, approximately 150, approximately 160, approximately 170, approximately 180, approximately 190 or approximately 200 or above on a 300 point scale may be used in selected embodiments to indicate which patients may respond favorably to the treatment methods of the instant invention (e.g., with a DLL3 ADC and/or chemotherapeutic agent). For example, in certain embodiments, a patient to be treated with an DLL3 ADC will have an ASCL1 H-score of at least 90 (i.e., the tumor is ASCL1⁺) on a 300 point scale. In other embodiments a patient to be treated with a DLL3 ADC as set forth in the instant invention will have an ASCL1 H-score of at least 120. In yet other embodiments a patient to be treated with the DLL3 ADCs of the instant invention will have an ASCL1 H-score of at least 180. For the purposes of the instant disclosure any tumor exhibiting an ASCL1 H-score of 90 or above on a 300 point scale will be considered ASCL1⁺and subject to treatment with any known therapy useful for treating neuroendocrine tumors, including targeting of transcriptional targets of ASCL1, as described further below.

In other selected embodiments an H-score comprising a 200 point scale may also be used to select or diagnose patients that may respond to treatments as disclosed herein. In such 200 H-score scales an H-score of 120 is approximately equivalent to an H-score of 180 on a 300 H-score scale. In both cases (e.g., 120/200 or 180/300) such H-scores may be classified as ASCL1⁺ (i.e., they are both above an H-score of 90 on a 300 point scale and/or ≥10% of the constituent cells express DLL3) and are suggestive of patients that may respond favorably to the treatment methods of the instant invention.

In other embodiments patient selection may be based on the measurement of percent of positively stained ASCL1 cells in a tumor sample. In this regard patients exhibiting a certain percentage of positively stained cells in an IHC sample when interrogated with an anti-ASCL1 antibody would be considered ASCL1⁺ and would be selected for treatment in accordance with the teachings herein. In such embodiments tumor samples exhibiting greater than 10%, greater than 20%, greater than 30%, greater than 40% or greater than 50% positive cell staining may be classified as ASCL1⁺ when measured as percent positive cells. In other embodiments tumor samples exhibiting greater than 60%, greater than 70%, greater than 80%, greater than 90% or greater than 95% positive cell staining may be classified as ASCL1⁺ when measured as percent positive. In certain preferred aspects the ASCL1⁺ tumor will express ASCL1 in ≥50% of the constituent cells when measured as percent positive. In each of the forgoing embodiments patients suffering from ASCL1⁺ tumors may be treated with DLL3 ADCs as set forth herein.

In still other embodiments patient selection may be predicated on the percent of ASCL1 positive cells staining with a certain intensity. By way of example, a tumor with >20% of the cells exhibiting 2+ intensity or greater will be a candidate for treatment with a DLL3 ADC. In other embodiments a patient will be a candidate for treatment with a DLL3 ADC or other chemotherapeutic agent if ≥20%, ≥30%, ≥40%, ≥50%, ≥60%, ≥70% or ≥80% of the tumor cells exhibit 1+intensity or greater when stained with an ASCL1 antibody and examined in accordance with standard IHC protocols as disclosed herein. In other certain embodiments a patient will be a candidate for treatment with a DLL3 ADC or other chemotherapeutic agent if ≥20%, ≥30%, ≥40%, ≥50%, ≥60%, ≥70% or ≥80% of the tumor cells exhibit 2+ intensity or greater when stained with an ASCL1 antibody and examined in accordance with standard IHC protocols as disclosed herein. In yet other selected embodiments a patient will be a candidate for treatment with a DLL3 ADC or other chemotherapeutic agent if ≥10%, >20%, ≥30%, ≥40% or ≥50% of the tumor cells exhibit 1+ intensity or greater when stained with an ASCL1 antibody and examined in accordance with standard IHC protocols as disclosed herein. In still other embodiments a patient will be a candidate for treatment with a DLL3 ADC or other chemotherapeutic agent if ≥10%, ≥20%, ≥30%, ≥40% or ≥50% of the tumor cells exhibit 2+ intensity or greater when stained with an ASCL1 antibody and examined in accordance with standard IHC protocols as disclosed herein. Yet another embodiment comprises a method of treating a subject having a tumor comprising tumor cells wherein ≥10% of the tumor cells exhibit 1+ intensity or greater when stained with an ASCL1 antibody and examined in accordance with standard IHC protocols comprising the step of administering an anti-DLL3 ADC. With regard to each of the aforementioned embodiments it will be appreciated that the intensity of staining with an ASCL1 antibody may be readily determined using standard pathology techniques and methodology familiar to those of skill in the art.

In yet other embodiments of the invention the expression of ASCL1 determinant may be measured using flow cytometry comprising fluorescent antibody staining. When using such techniques tumor cells may be defined as exhibiting positive, low and negative ASCL1 levels based fluorescent signals. Cells with negative expression (i.e. “FMO⁻”) may be defined as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one control”, or “FMO control”, staining. Cells with expression greater than the 95th percentile of expression observed with an isotype control antibody using the FMO staining procedure described above may be defined as “positive” (i.e. “FMO⁺”). As defined herein there are various populations of cells broadly defined as “positive” including those that may be defined as FMO⁺. A cell is defined as FMO⁺ if the mean observed expression of the antigen is above the 95th percentile determined using FMO staining with an isotype control antibody as described above. The positive cells may be termed cells with low expression (i.e. “FMO-low”) if the mean observed expression is above the 95th percentile determined by FMO staining and is within one standard deviation of the 95th percentile. Alternatively, the positive cells may be termed cells with high expression (i.e. “FMO-hi”) if the mean observed expression is above the 95th percentile determined by FMO staining and greater than one standard deviation above the 95th percentile. In other embodiments the 99th percentile may preferably be used as a demarcation point between negative and positive FMO staining. A sample that is FMO⁺ for a particular marker, for example, ASCL1⁺, has a detectable level of expression for the marker as compared to a control sample. A tumor that is positive for a particular marker can have detectable levels of the marker in one or more cells.

Other particularly compatible aspects of the invention involve the use of in situ hybridization to detect or monitor ASCL1 determinants. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic ASCL1 determinants. Probes preferably hybridize to a nucleotide sequence that corresponds to such determinants. Hybridization conditions can be routinely optimized to minimize background signal by non-fully complementary hybridization though preferably the probes are preferably fully complementary to the selected ASCL1 determinant. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In certain embodiments the antibodies of the instant invention may be used to detect and quantify levels of a particular determinant (e.g., ASCL1 protein) in a patient sample (e.g., plasma or blood) which may, in turn, be used to detect, diagnose or monitor proliferative disorders that are associated with the relevant determinant. In related embodiments the antibodies of the instant invention may be used to detect, monitor and/or quantify circulating tumor cells either in vivo or in vitro (WO 2012/0128801). In still other embodiments the circulating tumor cells may comprise tumorigenic cells.

In another embodiment, the invention provides a method of analyzing cancer progression and/or pathogenesis in vivo. In another embodiment, analysis of cancer progression and/or pathogenesis in vivo comprises determining the extent of tumor progression. In another embodiment, analysis comprises the identification of the tumor. In another embodiment, analysis of tumor progression is performed on the primary tumor. In another embodiment, analysis is performed over time depending on the type of cancer as known to one skilled in the art. In another embodiment, further analysis of secondary tumors originating from metastasizing cells of the primary tumor is conducted in vivo. In another embodiment, the size and shape of secondary tumors are analyzed. In some embodiments, further ex vivo analysis is performed.

In another embodiment, the invention provides a method of analyzing cancer progression and/or pathogenesis in vivo including determining cell metastasis or detecting and quantifying the level of circulating tumor cells. In yet another embodiment, analysis of cell metastasis comprises determination of progressive growth of cells at a site that is discontinuous from the primary tumor. In some embodiments, procedures may be undertaken to monitor tumor cells that disperse via blood vasculature, lymphatics, within body cavities or combinations thereof. In another embodiment, cell metastasis analysis is performed in view of cell migration, dissemination, extravasation, proliferation or combinations thereof.

In certain examples, the tumorigenic cells in a subject or a sample from a subject may be assessed or characterized using the disclosed antibodies prior to therapy to establish a baseline or select patients. In other examples the sample is derived from a subject that was treated. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the tumorigenic cells are assessed or characterized after a certain number of doses (e.g., after 2, 5, 10, 20, 30 or more doses of a therapy). In other examples, the tumorigenic cells are characterized or assessed after 1 week, 2 weeks, 1 month, 2 months, 1 year, 2 years, 3 years, 4 years or more after receiving one or more therapies.

B. Indications

The compositions and methods of the invention are useful in identifying, detecting or diagnosing proliferative disorders (e.g., cancer) expressing ASCL1 and, potentially, downstream therapeutic targets such as DLL3. Such detection and diagnosis will aid in the classification and etiology of the disorder and may suggest certain therapeutic compositions, such as DLL3 ADCs, that may be particularly effective in the treatment of the patient. Preferably the “subject” or “patient” to be treated will be human although, as used herein, the terms are expressly held to comprise any mammalian species.

In certain aspects the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In other preferred embodiments the disclosed ASCL1 antibodies are particularly effective in diagnosing patients having treating pancreatic cancer and, in selected aspects, lung adenocarcinoma. In certain embodiments the lung cancer is refractory, relapsed or resistant to an anthracyclines and/or a taxane (e.g., docetaxel, paclitaxel, larotaxel or cabazitaxel). In still other aspects of the invention the disclosed antibodies may be used to indicate which patients are particularly suitable for DLL3 ADC treatment of medullary thyroid cancer, large cell neuroendocrine carcinoma (LCNEC), glioblastoma, neuroendocrine prostate cancer (MEPC), high-grade gastroenteropancreatic cancer (GEP) and malignant melanoma. In still other preferred embodiments the disclosed antibodies may be used to indicate patients in which DLL3 ADCs may be used to treat bladder cancer.

Exemplary proliferative disorders, include but are not limited to adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma).

In certain embodiments, the proliferative disorder comprises small cell lung cancer (SCLC) and non-small cell lung cancer (NSCLC) (e.g., squamous cell non-small cell lung cancer or squamous cell small cell lung cancer). In certain embodiments, the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel). In certain embodiments the proliferative disorder is a limited stage disease. In other embodiments the proliferative disorder is an extensive stage disease. In other embodiments the proliferative disorder is refractory (i.e., which recurs during or shortly after completing a course of initial therapy). In other embodiments the proliferative disorder occurs in a sensitive patient (i.e., those whose relapse is longer than 2-3 months after primary therapy). In each of the aforementioned examples the subject may be treated with DLL3 ADCs following detection, classification or diagnosis of the cancer using the ASCL1 antibodies of the instant invention.

In certain embodiments, the proliferative disorder comprises a tumor with neuroendocrine features or phenotypes including neuroendocrine tumors. True or canonical neuroendocrine tumors (NETs) arising from the dispersed endocrine system are relatively rare, with an incidence of 2-5 per 100,000 people, but highly aggressive. Neuroendocrine tumors occur in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). These tumors may secrete several hormones including serotonin and/or chromogranin A that can cause debilitating symptoms known as carcinoid syndrome. Such tumors can be denoted by positive immunohistochemical markers such as neuron-specific enolase (NSE, also known as gamma enolase, gene symbol=ENO2), CD56 (or NCAM1), chromogranin A (CHGA), and synaptophysin (SYP) or by genes known to exhibit elevated expression such as ASCL1. Unfortunately traditional chemotherapies have not been particularly effective in treating NETs and liver metastasis is a common outcome.

Pseudo neuroendocrine tumors (pNETs) that genotypically or phenotypically mimic, resemble or exhibit common traits with canonical neuroendocrine tumors. Pseudo neuroendocrine tumors or tumors with neuroendocrine features are tumors that arise from cells of the diffuse neuroendocrine system or from cells in which a neuroendocrine differentiation cascade has been aberrantly reactivated during the oncogenic process. Such pNETs commonly share certain phenotypic or biochemical characteristics with traditionally defined neuroendocrine tumors, including the ability to produce subsets of biologically active amines, neurotransmitters, and peptide hormones. Histologically, such tumors (NETs and pNETs) share a common appearance often showing densely connected small cells with minimal cytoplasm of bland cytopathology and round to oval stippled nuclei. Accordingly, as used herein, reference to neuroendocrine tumors is inclusive of pseudo neuroendocrine tumors as well.

The anti-ASCL1 antibody compositions may also be used to detect or diagnose tumors that are at risk of transitioning to a neuroendocrine phenotype. Accordingly, the anti-ASCL1 antibody compositions may be used to diagnose adenocarcinoma arising in the lung, prostate, bladder, kidney, genitourinary tract, including bladder, prostate, ovary, cervix, and endometrium; gastrointestinal tract, including colon, and stomach; thyroid, including medullary thyroid cancer; and lung, including small cell lung carcinoma and large cell neuroendocrine carcinoma. Again, in each case the patient may be treated with targeted therapies (e.g., DLL3 ADCs) following diagnosis or classification of the disorder.

The anti-ASCL1 antibody compositions may also be used to detect or diagnose tumors at risk of neuroendocrine transition in subjects that have undergone or are undergoing a targeted therapy, or to select subjects for undergoing a targeted therapy. In some embodiments, the anti-ASCL1 antibodies may be used to diagnose tumors that are resistant to a targeted therapy. Accordingly, the anti-ASCLI antibodies may be used to diagnose EGFR inhibitor resistant lung cancer, or castration resistant prostate cancer.

Accordingly the anti-ASCL1 antibody compositions of the invention may beneficially be used to diagnose both pseudo neuroendocrine tumors and canonical neuroendocrine tumors. In this regard the anti-ASCL1 antibody compositions may be used as described herein to diagnose neuroendocrine tumors (both NET and pNET) arising in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). Based on the teachings herein it may be desirable to treat such ASCL1 positive patients with DLL3 ADCs even though such patient tumors are not overtly DLL3 positive when tested using standard diagnostic techniques.

C. Articles of Manufacture

In certain embodiments, the invention relates to a packaged article comprising one or more containers or receptacles, e.g., an article of manufacture, such as an assay and/or diagnostic kit, comprising any of the anti-ASCL1 antibody composition(s) of the invention, optionally with a label(s) and/or with instructions for use. Such label(s) include(s) ingredients, amounts or dosages, and/or indications. Such instructions include directing or promoting, including advertising, use of said article of manufacture. In certain other embodiments, the packaged article contains a detectable amount of an anti-ASCL1 antibody, with or without an associated reporter molecule and optionally one or more additional agents for the detection, quantitation and/or visualization of cancerous cells. The kits contemplated by the invention can also contain appropriate reagents to combine the antibody of the invention with a diagnostic or therapeutic agent (e.g., see U.S. Pat. No. 7,422,739).

When the components of the kit are provided in one or more liquid solutions, the liquid solution can be non-aqueous, though typically an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the antibody of the invention in combination with additional diagnostic markers or agents, the solution may be pre-mixed, either in a molar equivalent combination, or with one component in excess of the other. Alternatively, the antibody of the invention and any optional diagnostic agent or other agent can be maintained separately within distinct containers prior to use.

In certain preferred embodiments the aforementioned kits comprising compositions of the invention will comprise a label, marker, package insert, bar code and/or reader indicating that the kit contents may be used for the treatment, prevention, detection, prognosis and/or diagnosis of cancer. In other preferred embodiments the kit may comprise a label, marker, package insert, bar code and/or reader indicating that the kit contents may be administered in accordance with a certain protocol or regimen to diagnose a subject suffering from cancer. In a particularly preferred aspect the label, marker, package insert, bar code and/or reader indicates that the kit contents may be used for the treatment, prevention, detection, prognosis and/or diagnosis of a hematologic malignancy (e.g., AML) or provide a protocol or regimen for diagnosis of the same. In other particularly preferred aspects the label, marker, package insert, bar code and/or reader indicates that the kit contents may be used for the treatment, prevention, detection, prognosis and/or diagnosis of lung cancer (e.g., adenocarcinoma).

Suitable containers include, for example, bottles, vials, syringes, etc. The containers can be formed from a variety of materials such as glass or pharmaceutically compatible plastics. In certain embodiments the container(s) can comprise a sterile access port, for example, the container may be an intravenous solution bag or a vial having a stopper that can be pierced by a hypodermic injection needle.

In some embodiments the kit can contain a means by which to dispense the antibody and any optional components, e.g., one or more needles or syringes (pre-filled or empty), a dropper, pipette, or other such like apparatus, from which the formulation may be injected or introduced onto a detection surface (e.g., a slide or a microtiter plate), or into a subject for collecting a tumor sample, or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

In certain embodiments, the invention relates to a method of manufacturing an article of manufacture comprising any of the anti-ASCL1 antibody compositions of the invention described herein, packaging the composition to obtain an article of manufacture and instructing, directing or promoting the use of the article of manufacture for any of the uses described herein. Such instructing, directing or promoting includes advertising.

In certain embodiments, any of the assays, including diagnostic kits or any article of manufacture comprising the anti-ASCL1 antibody compositions of the invention, may be used as a companion diagnostic.

In other embodiments the anti-ASCL1 antibody compositions of the instant invention may be used in conjunction with, or comprise, diagnostic or therapeutic compositions and/or devices useful in the prevention or treatment of proliferative disorders. For example, in on preferred embodiment the compounds and compositions of the instant invention may be combined with certain diagnostic devices or instruments that may be used to detect, monitor, quantify or profile cells or marker compounds involved in the etiology or manifestation of proliferative disorders. In certain other embodiments the marker compounds may comprise NSE, CD56, synaptophysin, chromogranin A, and PGP9.5.

In yet other embodiments the compositions and/or devices may be used to detect, monitor and/or quantify circulating tumor cells either in vivo or in vitro (see, for example, WO 2012/0128801 which is incorporated herein by reference). In still other preferred embodiments, and as discussed above, circulating tumor cells may comprise cancer stem cells.

D. Patient Selection

In other instances an in vitro diagnostic method using art-recognized procedures (e.g., immunohistochemistry (IHC), or RT-PCR) is performed. As described herein, ASCL1 is a transcription factor that controls expression of DLL3 and is an early marker of neuroendocrine transition. In certain embodiments, the present invention comprises a method for selecting a subject for anti-cancer therapy based on a diagnosis made comprising the anti-ASCL1 antibody compositions of the invention. As such, a preferred embodiment comprises a method of selecting a subject for treatment with an anti-DLL3 antibody drug conjugate by detecting ASCL1 expression in a tumor sample. Such a method can comprise (a) contacting an anti-ASCL1 antibody with a tumor sample obtained from the subject; (b) detecting ASCL1 antibodies bound to the tumor sample; and (c) selecting a subject having an ASCL1⁺ tumor sample for appropriate treatment, for example, with an anti-DLL3 antibody drug conjugate (ADC). Representative antibodies useful in the invention include those depicted in FIGS. 1A-1B, and variants of such antibodies as described herein above. The ASCL1 antibodies may be detected in the tumor sample by virtue of a detectable label bound to or otherwise associated with the antibody, or by use of secondary and/or tertiary antibodies to amplify the signal, as is well known in the art.

Appropriate treatments for ASCL1 positive patients include any cancer treatments known in the art, particularly those known to be effective for treating neuroendocrine disorders. Representative anti-cancer agents include, but are not limited to cytotoxic agents (or cytotoxins), cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Exemplary anti-cancer agents or cytotoxins (including homologs and derivatives thereof) useful in targeted therapies comprise 1-dehydrotestosterone, anthramycins, actinomycin D, bleomycin, calicheamicins (including n-acetyl calicheamicin), colchicin, cyclophosphamide, cytochalasin B, dactinomycin (formerly actinomycin), dihydroxy anthracin, dione, duocarmycin, emetine, epirubicin, ethidium bromide, etoposide, glucocorticoids, gramicidin D, lidocaine, maytansinoids such as DM-1 and DM-4 (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible targeted therapies comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and tubulysins, paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In further embodiments ADCs useful in conjunction with the teachings herein may comprise cytotoxins comprising therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I,) carbon (¹⁴C), copper (⁶²Cu, ⁶⁴Cu, ⁶⁷Cu), sulfur (³⁵S), radium (²²³R), tritium (³II), indium (115_{In,} ¹¹³In, ¹¹²_{In,} ¹¹¹In,) bismuth (²¹²³Bi ²¹³Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁸⁰Y, ⁴⁷Sc, ¹⁸⁶Re, ¹⁸⁸Re ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, ¹¹⁷Sn, ²²⁵ Ac, ⁷⁶Br, and ²¹¹At. Other radionuclides are also available as diagnostic and therapeutic agents, especially those in the energy range of 60 to 4,000 keV.

In other aspects of the invention, suitable therapies comprise antibody drug conjugates comprising a targeting antibody conjugated or otherwise associated with any of the foregoing anti-cancer agents.

### V. Miscellaneous

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

VI. References The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described method.

### EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Cloning and Expression of Recombinant ASCL1 Fusion Proteins and Engineering of Cell Lines Overexpressing ASCL1 Protein

DNA Fragments Encoding Human ASCL1 and ASCL2 Protein.

To generate certain materials of the present invention pertaining to the human ASCL1 (hASCL1) protein (GenBank accession NP_004307), a synthetic DNA fragment that encoded the open reading frame of the ASCL1 mRNA (GenBank accession NM_004316, nts 572-1282) was subcloned into a CMV driven expression vector in-frame and downstream of an IgK signal peptide sequence and upstream of either a 9-Histidine tag or a human IgG2 Fc cDNA, using standard molecular techniques. These CMV-driven expression vectors permit high level transient expression in HEK-293T and/or CHO-S cells. Suspension or adherent cultures of HEK-293T cells, or suspension CHO-S cells were transfected with these expression constructs, using polyethylenimine polymer as the transfecting reagent. Three to five days after transfection, the recombinant His-tagged or Fc-tagged proteins were purified from clarified cell-supernatants using an AKTA explorer and either Nickel-EDTA (Qiagen) or MabSelect SuRe™ Protein A (GE Healthcare Life Sciences) columns, respectively. Recombinant ASCL2 protein was created similarly, using a synthetic DNA fragment that encoded the open reading frame of the ASCL2 mRNA (GenBank accession NM_005170, nts 621-1202).

In other instances, a fragment of the ASCL1 protein was produced in a prokaryotic system (e.g., Escherichia coli) as follows. A synthetic DNA fragment codon-optimized for E. coli expression and encoding ASCL1 residues H79 to D178 (GenBank accession NM_004316, nts 809-1105), a protein sequence comprising the ASCL1 bHLH domain, was subcloned downstream and in frame with an N-terminal glutathione S-transferase tag in an Escherichia coli expression vector (pET-Duet1). The derivative pET-Duet1 constructs were transformed into BL21 cells, expression of the recombinant protein induced with IPTG, and the GST-ASCL1 fusion protein purified from inclusion bodies and refolded using standard molecular techniques.

To create a lentiviral vector plasmid encoding the hASCL1 protein, the synthetic DNA encoding the hASCL1 open reading frame was subcloned in-frame into the multiple cloning site (MCS) of a lentiviral expression vector pCDH-EF1-MCS-T2A-GFP (System Biosciences, Mountain View Calif.), which had been previously modified to introduce nucleotide sequences encoding a DDDK epitope tag upstream of the multiple cloning site (MCS). The T2A sequence downstream of the MCS promotes ribosomal skipping of a peptide bond condensation, resulting in expression of two independent proteins: high level expression of DDDK-tagged cell surface proteins encoded upstream of the T2A peptide, with co-expression of the GFP marker protein encoded downstream of the T2A peptide. This cloning step yielded the lentiviral vector plasmid pLMEGPA-hASCL1-NFlag.

Cell Line Engineering

Engineered cell lines overexpressing the hASCL1 protein were constructed using the pLMEGPA-hASCL1-NFlag lentiviral vector, described above, to transduce HEK-293T cell lines using standard lentiviral transduction techniques well known to those skilled in the art. hASCL1-positive cells were selected using fluorescent activated cell sorting (FACS) of high-expressing HEK-293T subclones (e.g., cells that were strongly positive for GFP, which serves as a surrogate for high intracellular expression of ASCL1 in cells).

### Example 2

### Generation of Murine ASCL1 Antibodies

Anti-ASCL1 murine antibodies were produced in two immunization campaigns as follows. For the first campaign mice from the strains Balb/c, CD-1, and FVB were inoculated with 10 μg hASCL1-Fc emulsified with an equal volume of TiterMax® adjuvant. Following the initial inoculation the mice were injected twice weekly for 4 weeks with 10 μg hASCL1 protein emulsified with an equal volume of alum adjuvant plus CpG.

Mice were sacrificed and draining lymph nodes (popliteal, inguinal, and medial iliac) were dissected and used as a source for antibody producing cells. A single-cell suspension of B cells was produced and (122.5'10⁶ cells) were fused with non-secreting SP2/0-Ag14 myeloma cells (ATCC #CRL-1581) at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum (Thermo #SH30080-03), 10% BM condimed (Roche #10663573001), 1 mM nonessential amino acids (Corning #25-025-CI) 1 mM HEPES Corning #25-060-CI), 100 IU penicillin-streptomycin (Corning #30-002-CI), 100 IU L-glutamine (Corning #25-005-CI) and were cultured in three T225 flasks containing 100 mL selection medium. The flasks were placed in a humidified 37° C. incubator containing 7% CO2 and 95% air for 6 days.

On day 6 after the fusion the hybridoma library cells were frozen-down temporarily. The cells were thawed in hybridoma selection medium and allowed to rest in a humidified 37° C. incubator for 1 day. The cells were sorted from the flask and plated at one cell per well (using a BD FACSAria I cell sorter) in 90 μL of supplemented hybridoma selection medium (as described above) into 12 Falcon 384-well plates. Remaining unused hybridoma library cells were frozen in liquid nitrogen for future library testing and screening.

The hybridomas were cultured for 10 days and the supernatants from 12×384 clones were screened by ELISA for antibodies specific to hASCL1 yet not cross-reactive with the family member hASCL2, using the following method. Plates were coated with purified hASCL1-Fc or hASCL2-Fc at 0.5 μg/mL in PBS buffer and incubated at 4° C. overnight. Plates were then washed with PBST and blocked with PBS with 5% FBS for 30 min. at 37° C. The blocking solution was removed and 15 μl PBST was added to the wells. 25 μl of hybridoma supernatant was added and incubated overnight at 4° C. After washing with PBST, 30 μL/well HRP-labeled goat anti-mouse IgG diluted 1:10,000 in PBSA was added for 30 min. at room temperature. The plates were washed and developed by the addition of 25 μL/well of the TMB substrate solution (Thermo Scientific) for approximately 5 min. at room temperature. An equal volume of 0.2 M H₂SO₄ was added to stop substrate development. The samples were then analyzed by spectrophotometer at OD 450. Supernatants that had an OD 450 greater than 3 times the background on the ASCL1 plate were considered to be positively reactive, while any clones that also had an OD 450 greater than 3 times the background on the ASCL2 plate were rejected as cross-reactive. Screening of these 12×384 clones from the hASCL1-Fc immunization campaigns yielded numerous antibodies that bound specifically to hASCL1, but not the related family member ASCL2, eighty of which were carried forward for further characterization.

A second immunization campaign was conducted similar to the first with the following modifications: 1) 299×10⁶ cells) were fused with non-secreting SP2/0-Ag14 myeloma cells (ATCC #CRL-1581) at a ratio of 1:1 by electro cell fusion using a model BTX Hybrimmune System (BTX Harvard Apparatus). On day 6, after the fusion, the hybridoma cells were not frozen and were sorted on that day. After the 10 day culture, 6×384 clones were screened by ELISA selecting for antibodies positive to hASCL1-GST (glutathione S-transferase) and counter screened against an irrelevant protein tagged with GST. As part of this, ALK phosphatase-labeled goat anti-mouse IgG diluted 1:5000 in PBSA was used instead of HRP-labeled goat anti-mouse IgG diluted 1:10:000. ELISA samples were analyzed at OD405 instead of OD450.

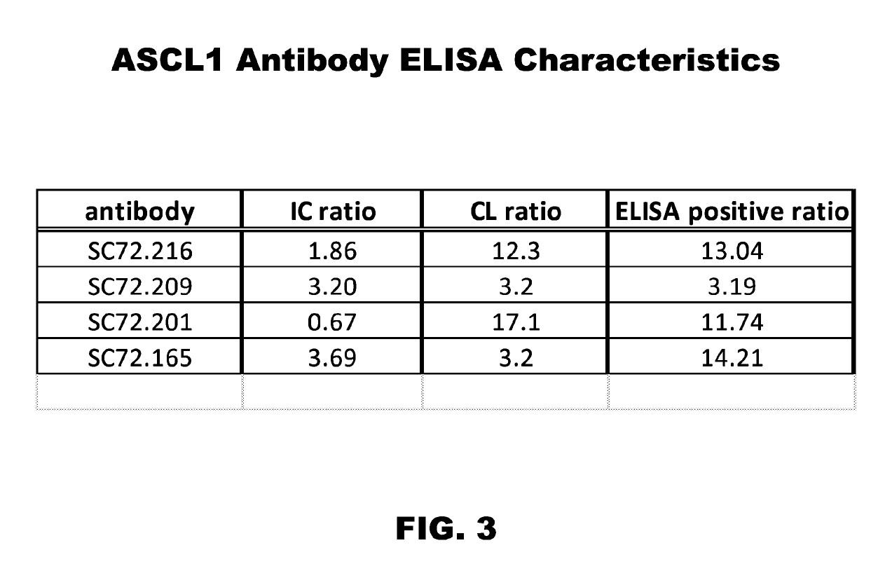

To confirm the results of the ELISA soup screen a similar ELISA was run on purified antibodies from selected clones. The ELISA positive ratio, (defined as ELISA staining against hASCL1 divided by the ELISA staining against an irrelevant protein) was then determined and set forth in FIG. 3 in the column labeled “ELISA positive ratio”.

It will be appreciated that the ELISA positive ratio values set forth in the column in FIG. 3 are indicative of antibodies that immunospecifically associate with a chosen target. As such this data, combined with the data from the hybridoma screen, indicates that the second hASCL1 immunization campaign also yielded a number of murine antibodies reactive with hASCL1.

### Example 3

### Sequencing of ASCL1 Antibodies

Based on the foregoing, a number of exemplary distinct monoclonal antibodies that bind immobilized human ASCL1 or h293-hASCL1 cells with apparently high affinity were selected for sequencing and further analysis. Sequence analysis of the light chain variable regions and heavy chain variable regions from selected monoclonal antibodies generated in Example 2 confirmed that many had novel complementarity determining regions and often displayed novel VDJ arrangements. The anti-ASCL1 mouse antibodies that were generated in Example 2 were sequenced as described below. Total RNA was purified from selected hybridoma cells using the RNeasy Miniprep Kit (Qiagen) according to the manufacturer's instructions. Between 10⁴ and 10⁵ cells were used per sample. Isolated RNA samples were stored at −80° C. until used.

The variable region of the Ig heavy chain of each hybridoma was amplified using two 5′ primer mixes comprising eighty-six mouse specific leader sequence primers designed to target the complete mouse VH repertoire in combination with a 3′ mouse Cγ primer specific for all mouse Ig isotypes. Similarly, two primer mixes containing sixty-four 5′ V_{κ} leader sequences designed to amplify each of the V_{κ} mouse families was used in combination with a single reverse primer specific to the mouse kappa constant region in order to amplify and sequence the kappa light chain. The VH and VL transcripts were amplified from 100 ng total RNA using the Qiagen One Step RT-PCR kit as follows. A total of four RT-PCR reactions were run for each hybridoma, two for the V_{κ} light chain and two for the VH heavy chain. PCR reaction mixtures included 1.5 μL of RNA, 0.4 μL of 100 μM of either heavy chain or kappa light chain primers (custom synthesized by Integrated DNA Technologies), 5 μL of 5× RT-PCR buffer, 1 μL dNTPₛ, and 0.6 μL of enzyme mix containing reverse transcriptase and DNA polymerase. The thermal cycler program was RT step 50° C. for 60 min., 95° C. for 15 min. followed by 35 cycles of (94.5° C. for 30 seconds, 57° C. for 30 seconds, 72° C. for 1 min.). There was then a final incubation at 72° C. for 10 min.

The extracted PCR products were sequenced using the same specific variable region primers as described above for the amplification of the variable regions. PCR products were sent to an external sequencing vendor (MCLAB) for PCR purification and sequencing services. Nucleotide sequences were analyzed using the IMGT sequence analysis tool available online at the website identified as http://www.imgt.org/IMGTmedical/sequence_analysis.html to identify germline V, D and J gene members with the highest sequence homology. The derived sequences were compared to known germline DNA sequences of the Ig V- and J-regions by alignment of VH and VL genes to the mouse germline database using a proprietary antibody sequence database.

FIG. 1A depicts the contiguous amino acid sequences of novel murine light chain variable regions from anti-ASCL1 antibodies while FIG. 1B depicts the contiguous amino acid sequences of novel murine heavy chain variable regions from the same anti-ASCL1 antibodies. Taken together murine light and heavy chain variable region amino acid sequences are provided in SEQ ID NOS: 21-73 odd numbers.

More particularly FIGS. 1A and 1B provide the annotated sequences of murine anti-ASCL1 antibodies comprising: (1) a light chain variable region (VL) of SEQ ID NO: 21 and a heavy chain variable region (VH) of SEQ ID NO: 23; or (2) a VL of SEQ ID NO: 25 and a VH of SEQ ID NO: 27; or (3) a VL of SEQ ID NO: 29 and a VH of SEQ ID NO: 31; or (4) a VL of SEQ ID NO: 33 and a VH of SEQ ID NO: 35; or (5) a VL of SEQ ID NO: 37 and a VH of SEQ ID NO: 39; or (6) a VL of SEQ ID NO: 41 and a VH of SEQ ID NO: 43; or (7) a VL of SEQ ID NO: 45 and a VH of SEQ ID NO: 47; or (8) a VL of SEQ ID NO: 49 and a VH of SEQ ID NO: 51; or (9) a VL of SEQ ID NO: 53 and a VH of SEQ ID NO: 55; or (10) a VL of SEQ ID NO: 57 and a VH of SEQ ID NO: 59; or (11) a VL of SEQ ID NO: 61 and a VH of SEQ ID NO: 63; or (12) a VL of SEQ ID NO: 65 and a VH of SEQ ID NO: 67; or (13) a VL of SEQ ID NO: 69 and a VH of SEQ ID NO: 71; or (14) a VL of SEQ ID NO: 21 and a VH of SEQ ID NO: 73.

A summary of the disclosed antibodies (or clones producing them), with their respective designation (e.g., SC72.2, SC72.28, etc.) and variable region nucleic acid or amino acid SEQ ID NOS (see FIGS. 1A-1C) are shown immediately below in Table 2.

TABLE 2

| | VL | VH |
|---|---|---|
| | SEQ ID NO: | SEQ ID NO: |
| Clone | NA/AA | NA/AA |
| SC72.2 | 20/21 | 22/23 |
| SC72.28 | 24/25 | 26/27 |
| SC72.52 | 28/29 | 30/31 |
| SC72.63 | 32/33 | 34/35 |
| SC72.76 | 36/37 | 38/39 |
| SC72.91 | 40/41 | 42/43 |
| SC72.94 | 44/45 | 46/47 |
| SC72.96 | 48/49 | 50/51 |
| SC72.132 | 52/53 | 54/55 |
| SC72.165 | 56/57 | 58/59 |
| SC72.181 | 60/61 | 62/63 |
| SC72.201 | 64/65 | 66/67 |
| SC72.216 | 68/69 | 70/71 |
| SC72.93 | 20/21 | 72/73 |

The VL and VH amino acid sequences in FIGS. 1A and 1B are annotated to identify the framework regions (i.e. FR1-FR4) and the complementarity determining regions (i.e., CDR-L1- CDR-L3 in FIG. 1A or CDR-H1-CDR-H3 in FIG. 1B), defined as per Kabat et al. The variable region sequences were analyzed using a proprietary version of the Abysis database to provide the CDR and FR designations. Though the CDRs are defined as per Kabat et al., those skilled in the art will appreciate that the CDR and FR designations can also be defined according to Chothia, McCallum or any other accepted nomenclature system. In addition, FIG. 1C provides the nucleic acid sequences (SEQ ID NOS: 20-72, even numbers) encoding the amino acid sequences set forth in FIGS. 1A and 1B.

As seen in FIGS. 1A and 1B and Table 2, the SEQ ID NOS. of the heavy and light chain variable region amino acid sequences for each particular murine antibody are generally sequential odd numbers. Thus, the monoclonal anti-ASCL1 antibody SC72.2 comprises amino acid SEQ ID NOS: 21 and 23 for the light and heavy chain variable regions respectively; SC72.28 comprises SEQ ID NOS: 25 and 27; SC72.52 comprises SEQ ID NOS: 29 and 31, and so on. The single exception to the sequential numbering scheme is SC72.93 which comprises the same light chain variable region amino acid sequence as clone SC72.2 (SEQ ID NO: 21) along with a unique heavy chain variable region amino acid sequence (SEQ ID NO: 73). In any event the corresponding nucleic acid sequence encoding the murine antibody amino acid sequence (set forth in FIG. 1C) has a SEQ ID NO. immediately preceding the corresponding amino acid SEQ ID NO. Thus, for example, the SEQ ID NOS. of the nucleic acid sequences of the VL and VH of the SC72.2 antibody are SEQ ID NOS: 20 and 22, respectively.

In addition to the annotated sequences in FIGS. 1A-1C, FIGS. 1D-1F provide, respectively, CDR designations for the light and heavy chain variable regions of SC72.165, SC72.181, and SC72.216 as determined using Kabat, Chothia, ABM and Contact methodology. The CDR designations depicted in FIGS. 1D-1F were derived using a proprietary version of the Abysis database as discussed above. Those of skill in the art will appreciate that the disclosed murine CDRs may be grafted into murine or human framework sequences to provide CDR grafted or humanized anti-ASCL1 antibodies in accordance with the instant invention. Moreover, in view of the instant disclosure one could readily determine the CDRs of any anti-ASCL1 antibody made and sequenced in accordance with the teachings herein and use the derived CDR sequences to provide CDR grafted or humanized anti-ASCL1 antibodies of the instant invention. This is particularly true of the antibodies with the heavy and light chain variable region sequences set forth in in FIGS. 1A-1B.

### Example 4

### Expression of ASCL1 mRNA in PDX Tumors

To characterize the cellular heterogeneity of solid tumors as they exist in cancer patients and to elucidate molecular subtypes within given tumor indications, a PDX tumor bank was developed and maintained using art recognized techniques. The PDX tumor bank, comprising a large number of discrete tumor cell lines, was propagated in immunocompromised mice through multiple passages of tumor cells originally obtained from cancer patients afflicted by a variety of solid tumor malignancies. Low passage PDX tumors are representative of tumors in their native environments, providing clinically relevant insight into underlying mechanisms driving tumor growth and resistance to current therapies. Selected PDX cell lines of pancreatic, colorectal and lung tumors were then interrogated as set forth immediately below.

In order to perform ASCL1 microarray analysis, PDX tumors were resected from mice after they reached 800-2,000 mm³. Resected PDX tumors were dissociated into single cell suspensions using art-recognized enzymatic digestion techniques (see, for example, U.S.P.N. 2007/0292414). Dissociated bulk tumor cells were incubated with 4′,6-diamidino-2-phenylindole (DAPI) to detect dead cells, anti-mouse CD45 and H-2K^{d} antibodies to identify mouse cells and anti-human EPCAM antibody to identify human cells. RNA was extracted from tumor cells by lysing the cells in RLTplus RNA lysis buffer (Qiagen) supplemented with 1% 2-mercaptoethanol, freezing the lysates at −80° C. and then thawing the lysates for RNA extraction using an RNeasy isolation kit (Qiagen). RNA was quantified using a Nanodrop spectrophotometer (Thermo Scientific) and/or a Bioanalyzer 2100 (Agilent Technologies). Normal tissue RNA was purchased from various sources (Life Technology, Agilent, ScienCell, BioChain, and Clontech). The resulting total RNA preparations were assessed by microarray analysis (Agilent Technologies).

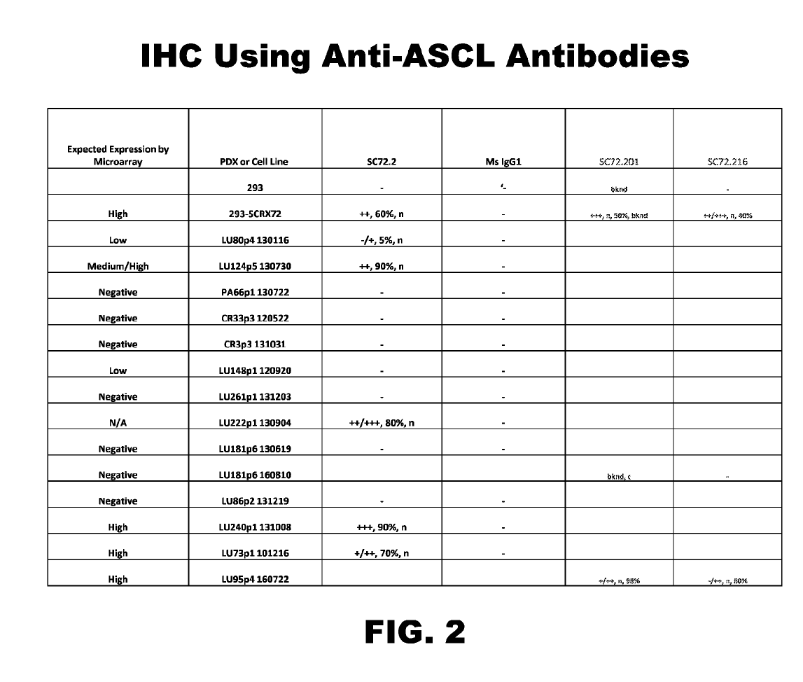

Microarray experiments on various PDX lines (and engineered 293 cell controls) were conducted and data was analyzed as follows: 1-2 μg of whole tumor total RNA was extracted, substantially as described above, from PDX lines. The RNA samples were analyzed using the Agilent SurePrint GE Human 8×60 v2 microarray platform, which contains 50,599 biological probes designed against 27,958 genes and 7,419 lncRNAs in the human genome. Standard industry practices were used to normalize and transform the intensity values to quantify gene expression for each sample. The normalized intensity of ASCL1 expression is set forth in column 1 of FIG. 2.

As shown in FIG. 2 the microarray analysis demonstrates that ASCL1 is expressed in a number of lung cancer cell lines indicating that it may provide a marker for detecting, diagnosing or monitoring certain neoplastic disorders.

### Example 5

### Expression of ASCL1 by IHC

To confirm the microarray data shown in the previous Example and further characterize the cellular heterogeneity of solid tumors as they exist in cancer patients, immunohistochemistry (IHC) assays were performed on PDX tumor sections to assess the expression of ASCL1 in tumor cells.

For exemplary antibody SC72.2 from the first immunization campaign, IHC was performed on formalin fixed and paraffin embedded (FFPE) tissues as is standard in the art. Planar sections of tissues were cut and mounted on glass microscope slides. After xylene de-paraffinization 5 μm sections were pre-treated with Antigen Retrieval Solution (Dako) for 20 mins. at 99° C., cooled to 75° C. and then treated with 0.3% hydrogen peroxide in PBS followed by Avidin/Biotin Blocking Solution (Vector Laboratories). FFPE slides were then blocked with 10% horse serum in 3% BSA in PBS buffer and incubated with a primary anti-ASCL1 antibody (clone SC72.2), diluted to 10 μg/ml in 3% BSA/PBS, for 30 mins. at room temperature. For exemplary antibodies from the second campaign (SC72.201 and SC72.216), IHC was performed as above except that the primary antibody incubation was for 1 hr as opposed to 30 mins.

The FFPE slides were then incubated with biotin-conjugated horse anti-mouse antibody (Vector Laboratories), diluted to 2.5 μg/ml in 3% BSA/PBS, for 30 mins. at room temperature followed by incubation in streptavidin-HRP (ABC Elite Kit; Vector Laboratories). Chromogenic detection was developed with 3,3′-diaminobenzidine (Thermo Scientific) for 5 min. at room temperature and tissues were counterstained with Meyer's hematoxylin (IHC World), washed with alcohol and immersed in xylene. Sections were then viewed by brightfield microscopy and ASCL1 expression was noted. Results of the studies are shown in a tabular form in the four left hand columns of FIG. 2.

FIG. 2 confirms that the disclosed antibodies may be used to effectively generate IHC data on ASCL1 engineered cells and patient derived tumor xenograft (PDX) cell lines expressing human ASCL1 protein. Moreover, the IHC data generally correlates with the ASCL1 expression as measured by microarray analysis further demonstrating that ASCL1 expression is associated with tumors and provides a viable diagnostic or prognostic target for management of the same.

### Example 6

### Intracellular FACS Staining of ASCL1

Selected anti-hASCL1 murine antibodies produced in Example 2 were also analyzed by flow cytometry to further elucidate their binding characteristics and provide additional confirmation that such antibodies may be used as diagnostic agents. To this end exemplary antibody clones were cultured in 2 mL of media and the secreted antibodies were then purified. The purified antibodies were then characterized by intracellular FACS staining with antibodies specific to hASCL1 as follows.

1×10⁵ per well of HEK293T cells transiently transduced with hASCL1(as described in Example 1), and HEK293T cells transiently transduced with GFP only were washed with cold PBS and then incubated with a solution of PBS and a Dylight 450 Fixable viability dye for 30 minutes. After incubation, the cells were washed twice with PBS. To permeate the cell walls 100 ul of BD Cytofix/Cytoperm was added to the cells and left to incubate for 20 minutes on ice. Cells were then washed with BD Phosflow perm/wash solution. Purified antibody at a final concentration of 10 ug/ml was then added to the cells in 45 ul of BD Phosflow perm/wash solution, incubated on ice for 30 minutes, and then washed with BD Phosflow perm/wash 2 times. 50 ul of Alexa Fluor 647 conjugated Goat-anti-mouse IgG secondary antibody, was added at 1 ug/ml dissolved in BD Phosflow perm/wash and incubated on ice for 30 minutes, then washed with BD Phosflow perm/wash 3 times. The cells were then resuspended in FACS buffer to be read on BD FACS Canto according to the manufacturer's instructions.

The FlowJo software package (FlowJo LLC) was used to calculate gMFI (geometric Mean Fluorescence Intensity) for each of the anti-ASCL1 antibodies. The exemplary antibodies exhibited calculated intracellular gMFI values (the column labeled “IC Ratio” in FIG. 3) indicating that they were binding to intracellular hASCL1 in HEK293T cells transiently transduced with hASCL1 but not HEK293T cells transiently transduced with GFP only.

These measurements further demonstrate that the disclosed antibodies selectively bind to ASCL1 and provide potential diagnostic agents for neoplastic disorders.

### Example 7

### Anti-ASCL1 Antibodies Bind ASCL1 in Cell Lysates

To further confirm binding specificity of the disclosed ASCU antibodies, a cell lysate MSD ELISA was performed using selected clones from Example 2.

293T cells transiently transfected with ASCL1 protein (as described above) were harvested from a T150 tissue culture flask, pelleted by centrifugation, and washed twice in cold PBS. Protein Extraction Buffer (Biochain Institute) was added to the cell pellet to lyse the cells. Lysates were cleared by centrifugation (20,000 g, 20 min., 4° C.) and the total protein concentration was quantified using bicinchoninic acid. Lysate from 293 cells transduced with a control protein were prepared in a similar manner to serve as a negative control. ASCL1 or negative control protein lysate was diluted to 40 ug/ml in PBS and added to the MSD 384-well standard plates at 15 μL/well. The plates were sealed and incubated overnight at 4° C. The following day, the plate content was flicked and replaced with 35 μL of PBS-containing 3% BSA for 1 hour at room temperature. ASCL1 antibodies were diluted to 2 ug/mL in PBS with 0.05% Tween 20 and 1% BSA (PBSTA buffer). After washing the blocked plates three times with PBST wash buffer, diluted antibodies were added to the plates at 10 μL/well in duplicate for 1 hour. A goat anti-mouse IgG, sulfo-tagged antibody was added to the washed plates at 0.5 μg/ml in 10 μL/well in PBSTA. Plates were then washed in PBST wash buffer. MSD Read Buffer T with surfactant was diluted to 1× in water and 35 μl was added to each well. Plates were read on a MSD SECTOR Imager instrument (Meso Scale Discovery).

A ratio of the raw electrochemiluminescence values from the ASCL1 lysate/irrelevant protein were generated and reported in FIG. 3 as indicated in column “CL ratio”. A large value indicates specific binding to ASCL1 lysate over negative control lysate. Accordingly, the data in FIG. 3 further indicates that the tested antibodies selectively bind ASCL1 obtained from cells and may serve as a diagnostic agent in a clinical setting.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

We claim:

1. An isolated anti-ASCL1 antibody that comprises or competes for binding to a human ASCL1 protein with an antibody comprising:
a light chain variable region (VL) of SEQ ID NO: 21 and a heavy chain variable region (VH) of SEQ ID NO: 23; or
a VL of SEQ ID NO: 25 and a VH of SEQ ID NO: 27; or
a VL of SEQ ID NO: 29 and a VH of SEQ ID NO: 31; or
a VL of SEQ ID NO: 33 and a VH of SEQ ID NO: 35; or
a VL of SEQ ID NO: 37 and a VH of SEQ ID NO: 39; or
a VL of SEQ ID NO: 41 and a VH of SEQ ID NO: 43; or
a VL of SEQ ID NO: 45 and a VH of SEQ ID NO: 47; or
a VL of SEQ ID NO: 49 and a VH of SEQ ID NO: 51; or
a VL of SEQ ID NO: 53 and a VH of SEQ ID NO: 55; or
a VL of SEQ ID NO: 57 and a VH of SEQ ID NO: 59; or
a VL of SEQ ID NO: 61 and a VH of SEQ ID NO: 63; or
a VL of SEQ ID NO: 65 and a VH of SEQ ID NO: 67; or
a VL of SEQ ID NO: 69 and a VH of SEQ ID NO: 71; or
a VL of SEQ ID NO: 21 and a VH of SEQ ID NO: 73.

2. The anti-ASCL1 antibody of claim 1 comprising:
a light chain variable region and a heavy chain variable region, wherein the light chain variable region has three CDRs of a light chain variable region set forth as SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65 or SEQ ID NO: 69 and the heavy chain variable region has three CDRs of a heavy chain variable region set forth as SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO:59 and SEQ ID NO: 63, SEQ ID NO: 67, SEQ ID NO: 71 or SEQ ID NO: 73.

3. The anti-ASCLI antibody of claim 2 comprising:
a light chain variable region (VL) of SEQ ID NO: 21 and a heavy chain variable region (VH) of SEQ ID NO: 23; or
a VL of SEQ ID NO: 25 and a VH of SEQ ID NO: 27; or
a VL of SEQ ID NO: 29 and a VH of SEQ ID NO: 31; or
a VL of SEQ ID NO: 33 and a VH of SEQ ID NO: 35; or
a VL of SEQ ID NO: 37 and a VH of SEQ ID NO: 39; or
a VL of SEQ ID NO: 41 and a VH of SEQ ID NO: 43; or
a VL of SEQ ID NO: 45 and a VH of SEQ ID NO: 47; or
a VL of SEQ ID NO: 49 and a VH of SEQ ID NO: 51; or
a VL of SEQ ID NO: 53 and a VH of SEQ ID NO: 55; or
a VL of SEQ ID NO: 57 and a VH of SEQ ID NO: 59; or
a VL of SEQ ID NO: 61 and a VH of SEQ ID NO: 63; or
a VL of SEQ ID NO: 65 and a VH of SEQ ID NO: 67; or
a VL of SEQ ID NO: 69 and a VH of SEQ ID NO: 71; or
a VL of SEQ ID NO: 21 and a VH of SEQ ID NO: 73.

4. The anti-ASCL1 antibody of any one of claims 1-3, wherein the anti-ASCLI antibody comprises a monoclonal antibody.

5. The anti-ASCL1 antibody of any one of claims 1-4, wherein the anti-ASCLI antibody is a murine antibody.

6. The anti-ASCL1 antibody of any one of claims 1-4, wherein the anti-ASCLI antibody is selected from the group consisting of a chimeric antibody, a CDR-grafted antibody, and a humanized antibody.

7. A nucleic acid encoding all or part of an antibody of any one of claims 1-6.

8. A vector comprising a nucleic acid of claim 7.

9. A host cell comprising a nucleic acid of claim 7 or a vector of claim 8.

10. An anti-ASCL1 antibody of any one of claims 1-6 wherein the antibody is conjugated to a detectable label.

11. A pharmaceutical composition comprising an antibody of any one of claims 1-6 or an antibody conjugate of claim 10.

12. A method of detecting ASCL1 in a subject, the method comprising:
(a) contacting a tumor sample obtained from a subject with an ASCL1 antibody; and
(b) detecting the ASCL1 antibody bound to the tumor sample, wherein the ASCLI antibody comprises or competes with an antibody of claim 1.

13. The method of claim 12 wherein the anti-ASCL1 antibody comprises a monoclonal antibody.

14. The method of claim 12 or 13 wherein the anti-ASCL1 antibody comprises a murine antibody.

15. The method of claim 12 or 13 wherein the anti-ASCL1 antibody is selected from the group consisting of a chimeric antibody, a CDR-grafted antibody, and a humanized antibody.

16. The method of any one of claims 12 to 15, wherein detecting the ASCL1 antibody is performed using immunohistochemistry.

17. The method of claim 16, wherein the tumor sample is chemically fixed.

18. The method of claim 17, wherein the tumor sample is chemically fixed using formalin.

19. The method of any one of claims 16-18, wherein the tumor is paraffin embedded.

20. The method of any one of claims 12-19, wherein the tumor is characterized by a poorly differentiated neuroendocrine phenotype or is at risk of transitioning to a neuroendocrine phenotype.

21. The method of claim 20, wherein the tumor occurs in lung, prostate, breast, ovary, genitourinary tract, gastrointestinal tract, thyroid or kidney.

22. The method of claim 21, wherein the tumor comprises lung cancer.

23. The method of claim 22, wherein the tumor comprises small cell lung cancer.

24. The method of claim 22, wherein the tumor comprises large cell neuroendocrine carcinoma.

25. The method of claim 21, wherein the tumor comprises ovarian cancer

26. The method of claim 21, wherein the tumor comprises medullary thyroid cancer.

27. The method of claim 21, wherein the tumor comprises renal cancer.

28. The method of claim 21, wherein the tumor comprises prostate cancer.

29. The method of claim 28, wherein the prostate cancer comprises castration resistant prostate cancer.

30. The method of claim 28, wherein the prostate cancer is resistant to androgen resistant therapy.

31. The method of any one of claims 12-30, wherein the ASCL1 antibody is conjugated or otherwise associated with a detectable label.

32. The method of any one of claims 12-30, wherein the ASCL1 antibody is unlabeled.

33. The method of claim 32, wherein detecting the ASCL1 antibody further comprises the steps of:
(c) contacting the tumor sample of (b) with an antibody that specifically binds to the ASCL1 antibody; and
(d) detecting the antibody that specifically binds to the ASCL1 antibody.

34. The method of any one of claims 12 to 33 further comprising the step of administering an anti-DLL3 antibody drug conjugate to the subject

35. An article of manufacture comprising one or more receptacles containing a pharmaceutical composition of claim 11.

36. The article of manufacture of claim 35 further comprising a label or package insert associated with the one or more receptacles indicating that the composition is for diagnosing cancer.

37. A method of detecting, diagnosing, or monitoring cancer in a subject, the method comprising the steps of (a) contacting tumor cells with an antibody of any one of claims 1-6; and (b) detecting the antibody on tumor cells.

38. The method of claim 37, wherein the contacting is performed in vitro.

39. The method of claim 37 wherein the contacting is performed in vivo.


SEQUENCE LISTING (73 sequences)

SEQ ID NO: 1 - protein, 236 aa, Homo sapiens
  MISC_FEATURE: NP_004307.2 achaete-scute homolog 1 [Homo sapiens]
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 2 - protein, 107 aa, Mus musculus
  MISC_FEATURE: Murine kappa light chain constant region
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 106 aa, Mus musculus
  MISC_FEATURE: Murine lambda light chain constant region
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 4 - protein, 324 aa, Mus musculus
  MISC_FEATURE: IgG1 murine heavy chain constant region
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 5-19 - skipped (15 entries)

SEQ ID NO: 20 - DNA, 339 bp, Mus musculus
  misc_feature: SC72.2 & SC72.93 Light Chain Variable Region
gatgttgtgatgacccagactccactcactttgtcggttaccattggacaaccagcttccatctcttgcaagtcaagtcagagcctcttagatagtgatggaaaaacctatttaaattggttattacagaggccaggcgagtctccaaagctcctaatctatctggtgtctaaactggactctggagtccctgacaggttcactggcagtggatcagggacagatttcacactgaaaatcagcagagtggaggctgaggatttgggagtttattactgcttgcaagctacacattttcctcagctcacgttcggtgctgggaccaagctggagctgaaa

SEQ ID NO: 21 - protein, 113 aa, Mus musculus
  MISC_FEATURE: SC72.2 & SC72.93 Light Chain Variable Region
DVVMTQTPLTLSVTIGQPASISCKSSQSLLDSDGKTYLNWLLQRPGESPKLLIYLVSKLDSGVPDRFTGSGSGTDFTLKISRVEAEDLGVYYCLQATHFPQLTFGAGTKLELK

SEQ ID NO: 22 - DNA, 342 bp, Mus musculus
  misc_feature: SC72.2 Heavy Chain Variable Region
caggttcaactgcagcagtctggggctgagttggtgaggcctggggcttcagtgacgctgtcctgcaaggcttcgggctacacatttattgactatgaaatgcactgggtgaagcagacacctgtgcatggcctggaatggattggagctattgatcctgaaactggtggtactacctacaatcagaacttcaagggcaaggccacactgactgcagacaaatcctccaacacagcctacatggagctccgcagcctgacatctggggactctgccgtctattactgttcaagagggggtcttgactactggggccaaggcaccactctcacagtctcctca

SEQ ID NO: 23 - protein, 114 aa, Mus musculus
  MISC_FEATURE: SC72.2 & SC72.93 Heavy Chain Variable Region
QVQLQQSGAELVRPGASVTLSCKASGYTFIDYEMHWVKQTPVHGLEWIGAIDPETGGTTYNQNFKGKATLTADKSSNTAYMELRSLTSGDSAVYYCSRGGLDYWGQGTTLTVSS

SEQ ID NO: 24 - DNA, 336 bp, Mus musculus
  misc_feature: SC72.28 Light Chain Variable Region
gatgttgtgatgacccagactccactcactttgtcggttacctttggacaaccagcttccatctcttgtaagtcaagtcagagcctcttagatagtgatggaaaaacctatttaaattggttattacagaggccaggcgagtctccaaagctcctaatctatctggtgtctaaactggactctggagtccctgacaggttcactggcagtggatcagggacagatttcacactgaaaatcagcagagtggaggctgaggatttgggagtttattactgcttgcaagctacacattttcctctcacgttcggtgctgggaccaagctggagctgaaa

SEQ ID NO: 25 - protein, 112 aa, Mus musculus
  MISC_FEATURE: SC72.28 Light Chain Variable Region
DVVMTQTPLTLSVTFGQPASISCKSSQSLLDSDGKTYLNWLLQRPGESPKLLIYLVSKLDSGVPDRFTGSGSGTDFTLKISRVEAEDLGVYYCLQATHFPLTFGAGTKLELK

SEQ ID NO: 26 - DNA, 342 bp, Mus musculus
  misc_feature: SC72.28 Heavy Chain Variable Region
gacgttcagctgcagcagtctggggctgagcttgtgaggccaggggccttagtcaagttgtcctgcaaagcttctggcttcaacattaaagactaccttatacactgggtgaagcagaggcctgaacagggcctggagtggattggatgtattgatcctgagaatggtaatactatatatgacccgaggttccagggcaaggccaaaataacatcagacacatcctccaccacagcctacctccggctcaccagcctgacatctgaggacactgccgtctattactgtggtagggggggccttgactactggggccaaggcaccactctcacagtctcctca

SEQ ID NO: 27 - protein, 114 aa, Mus musculus
  MISC_FEATURE: SC72.28 Heavy Chain Variable Region
DVQLQQSGAELVRPGALVKLSCKASGFNIKDYLIHWVKQRPEQGLEWIGCIDPENGNTIYDPRFQGKAKITSDTSSTTAYLRLTSLTSEDTAVYYCGRGGLDYWGQGTTLTVSS

SEQ ID NO: 28 - DNA, 336 bp, Mus musculus
  misc_feature: SC72.52 Light Chain Variable Region
gatattgtgataacccaggatgaactctccaatcctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaatttgtagagtatccgctcacgttcggtgctgggaccaagctggagctgaaa

SEQ ID NO: 29 - protein, 112 aa, Mus musculus
  MISC_FEATURE: SC72.52 Light Chain Variable Region
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQFVEYPLTFGAGTKLELK

SEQ ID NO: 30 - DNA, 348 bp, Mus musculus
  misc_feature: SC72.52 Heavy Chain Variable Region
caggtccaactgcagcagcctgggtctgtgctggtgaggcctggagcttcagtgaagctgtcctgcaaggcttctggctacacattcaccagctactggatgcactgggtgaagcagaggcctggacaaggccttgagtggattggagagatttatcctaatagtggtaggactaactacaatgagaagttcaagggcaaggccacactgactgttgacacatcctccagcaccgcctacgtggatctcagcagcctgacatctgaggactctgcggtctattactgtcattactacgatgataactacggctactggggccaaggcaccactctcacagtctcctca

SEQ ID NO: 31 - protein, 116 aa, Mus musculus
  MISC_FEATURE: SC72.52 Heavy Chain Variable Region
QVQLQQPGSVLVRPGASVKLSCKASGYTFTSYWMHWVKQRPGQGLEWIGEIYPNSGRTNYNEKFKGKATLTVDTSSSTAYVDLSSLTSEDSAVYYCHYYDDNYGYWGQGTTLTVSS

SEQ ID NO: 32 - DNA, 321 bp, Mus musculus
  misc_feature: SC72.63 Light Chain Variable Region
gacatccagatgactcagtctccagcctccctatctgcatctgtgggagaaactgtcaccatgacatgtcgagcaagtgagaatatttacagtaatttagcatggtatcagcagaaacagggaaaatctcctcagctcctggtctatgctgcaacaaacttagcagatgatgtgccatcaaggttcagtggcagtggatcaggcacacagttttctctgaagatcaacagcctgcagcctgaagattttgggatttattactgtcatcatttttacggttctcctctcacgttcggtgctgggaccaagctggagctgaaa

SEQ ID NO: 33 - protein, 107 aa, Mus musculus
  MISC_FEATURE: SC72.63 Light Chain Variable Region
DIQMTQSPASLSASVGETVTMTCRASENIYSNLAWYQQKQGKSPQLLVYAATNLADDVPSRFSGSGSGTQFSLKINSLQPEDFGIYYCHHFYGSPLTFGAGTKLELK

SEQ ID NO: 34 - DNA, 357 bp, Mus musculus
  misc_feature: SC72.63 Heavy Chain Variable Region
gaggtccagctgctgcagtctggacctgagctggtgaagcctggggcttcagtgaagatgtcctgcaaggcttctggatacacactcactgactataacttgcactgggtgaagcagagccatgcaaagagccttgagtggattggatatattaacccttacaatggtgttacttcctacaaccagaggttcaagggcaaggccacattgactgtaaacaagtcctccagcacagccttcatggagttccgcagcctgacatcggaggattctgcagtctattactgtgtatctctctactatggtgacgacccgtttgcttgctggggccaagggactctggtcactgtctcaaca

SEQ ID NO: 35 - protein, 119 aa, Mus musculus
  MISC_FEATURE: SC72.63 Heavy Chain Variable Region
EVQLLQSGPELVKPGASVKMSCKASGYTLTDYNLHWVKQSHAKSLEWIGYINPYNGVTSYNQRFKGKATLTVNKSSSTAFMEFRSLTSEDSAVYYCVSLYYGDDPFACWGQGTLVTVST

SEQ ID NO: 36 - DNA, 333 bp, Mus musculus
  misc_feature: SC72.76 Light Chain Variable Region
gacattgtgctgacacagtctcctgcttccttaactgtatctctggggcagagggccaccatctcatgcagggccagccaaagtgtcagaacatctaactatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcaccattgggagattccgttcacgttcggaggggggaccaagctggaaataaaa

SEQ ID NO: 37 - protein, 111 aa, Mus musculus
  MISC_FEATURE: SC72.76 Light Chain Variable Region
DIVLTQSPASLTVSLGQRATISCRASQSVRTSNYSYMHWYQQKPGQPPKLLIKYASNLESGVPARFSGSGSGTDFTLNIHPVEEEDTATYYCQHHWEIPFTFGGGTKLEIK

SEQ ID NO: 38 - DNA, 342 bp, Mus musculus
  misc_feature: SC72.76 Heavy Chain Variable Region
caggtgcagctgaaggagtcaggacctggcctggtggcgccctcacagagcctgtccatcacctgcacagtctctgatttctcattaaccaactatgctgtacactgggttcgccagtctccaggaaagggtctggagtggctgggagtgatatggagtgatggaaacacagactataatgcgcctttcatatctagactgagcatcagcaaggacaactccaagagccaagttttctttaaaatgaacagtctgcaagctgatgacacagccatatactactgtgccagctatggtgattctgcttactggggccaagggactctggtcactgtctctgca

SEQ ID NO: 39 - protein, 114 aa, Mus musculus
  MISC_FEATURE: SC72.76 Heavy Chain Variable Region
QVQLKESGPGLVAPSQSLSITCTVSDFSLTNYAVHWVRQSPGKGLEWLGVIWSDGNTDYNAPFISRLSISKDNSKSQVFFKMNSLQADDTAIYYCASYGDSAYWGQGTLVTVSA

SEQ ID NO: 40 - DNA, 336 bp, Mus musculus
  misc_feature: SC72.91 Light Chain Variable Region
gatgttgtgctgacccaaactccactctccctgcctgtcagtcttggagatcaagcctccatttattgtagatctagtcagagcattgtacatagtaatggaaacacctatttcgaatggtacctgcagaaaccaggccagtctccaaagctcctgatctacaaagtttccaaccgattgtctggggtcccagacaggttcagtggcagtggttcagggacagacttcacactcaagatcagcagagtggaggctgttgatctgggaatttattactgctttcaagcttcacatgttccgttcacgttcggtgctgggaccaagctggagctgaaa

SEQ ID NO: 41 - protein, 112 aa, Mus musculus
  MISC_FEATURE: SC72.91 Light Chain Variable Region
DVVLTQTPLSLPVSLGDQASIYCRSSQSIVHSNGNTYFEWYLQKPGQSPKLLIYKVSNRLSGVPDRFSGSGSGTDFTLKISRVEAVDLGIYYCFQASHVPFTFGAGTKLELK

SEQ ID NO: 42 - DNA, 372 bp, Mus musculus
  misc_feature: SC72.91 Heavy Chain Variable Region
caggttcagctgcagcagtctggacctgaggtggtgaagcctggggcttcagtgaagttgtcctgcaaggcttctggctacaccttcacaacctacgatataaactgggtgaagcagaggcctggacagggacttgagtggattggatggatttattttagagatggtactaataagtacaatgagaagttcaagggcaaggccacattgactgtagacacatcctccagcacagcgtacatggagctccacagcctgacatctgaggactctgcggtctatttctgtgcaagcgtctatgatggttactacgtttcgggatttgatattatggactattggggtcaaggaacctcagtcaccgtttcctca

SEQ ID NO: 43 - protein, 124 aa, Mus musculus
  MISC_FEATURE: SC72.91 Heavy Chain Variable Region
QVQLQQSGPEVVKPGASVKLSCKASGYTFTTYDINWVKQRPGQGLEWIGWIYFRDGTNKYNEKFKGKATLTVDTSSSTAYMELHSLTSEDSAVYFCASVYDGYYVSGFDIMDYWGQGTSVTVSS

SEQ ID NO: 44 - DNA, 321 bp, Mus musculus
  misc_feature: SC72.94 Light Chain Variable Region
gacatccaaatgaaccagtctccatccagtctgtctgcatccctcggagacacaattaccatcacttgccgtgccagtcagggcattagtatttggttaagctggtaccagcagaaaccaggaaatattcctaaactattgatctataaggcttccaacttgcacacaggagtcccaccaagctttagtggcagtggatctggaacagatttcacattaactatcagcagcctacagcctgaagacattgccacttactactgtctacagagtcaaagttatccgtggacgttcggtgggggcaccaagctagaaatcaaa

SEQ ID NO: 45 - protein, 107 aa, Mus musculus
  MISC_FEATURE: SC72.94 Light Chain Variable Region
DIQMNQSPSSLSASLGDTITITCRASQGISIWLSWYQQKPGNIPKLLIYKASNLHTGVPPSFSGSGSGTDFTLTISSLQPEDIATYYCLQSQSYPWTFGGGTKLEIK

SEQ ID NO: 46 - DNA, 336 bp, Mus musculus
  misc_feature: SC72.94 Heavy Chain Variable Region
caggtccaactgcagcagcctgggactgaactggtgaagcctggggcttcagtgaagctgtcctgcaaggcttctgcctacaccttcaccagcctctggattcactgggtgaagcagaggcctggacaaggccttgagtggattggagatattaatcctagtattggtgatacttactacaatgagaggttcaagagcaaggccacactgactgtagacaaatcctccagcacagcctacatgcagctcagcagcctgacatctgaggactctgcggtttattattgtacaactggcgactcctggggccaaggcaccactctcacagtctcctca

SEQ ID NO: 47 - protein, 112 aa, Mus musculus
  MISC_FEATURE: SC72.94 Heavy Chain Variable Region
QVQLQQPGTELVKPGASVKLSCKASAYTFTSLWIHWVKQRPGQGLEWIGDINPSIGDTYYNERFKSKATLTVDKSSSTAYMQLSSLTSEDSAVYYCTTGDSWGQGTTLTVSS

SEQ ID NO: 48 - DNA, 321 bp, Mus musculus
  misc_feature: SC72.96 Light Chain Variable Region
gacatccagatgactcagtctccagcctccctatctgcatctgtgggagaaactgtcaccatgacatgtcgagcaagtgaaaatattcacagtaatttagtatggtatcagcagaaacagggaaaatctcctcaactcctggtctatgctgcaacaaacttagcagatggtgtgccatcaaggttcagtggcagtggttcaggctcacagttttctctgaagatcgacagcctgcagcctgaagattttgggagttatctctgtcaacatttttatggcactcctcgtaccttcggtggaggcaccaagctggaaatcaaa

SEQ ID NO: 49 - protein, 107 aa, Mus musculus
  MISC_FEATURE: SC72.96 Light Chain Variable Region
DIQMTQSPASLSASVGETVTMTCRASENIHSNLVWYQQKQGKSPQLLVYAATNLADGVPSRFSGSGSGSQFSLKIDSLQPEDFGSYLCQHFYGTPRTFGGGTKLEIK

SEQ ID NO: 50 - DNA, 360 bp, Mus musculus
  misc_feature: SC72.96 Heavy Chain Variable Region
gaggtccagctgcaacagtcaggacctgagctggtgaagcctggggcttcagtgaagatgtcctgcaaggcttctggatacacactcactgcctacaacatacactgggtgaagcagagccatggaaagagccttgagtggattggatttattaaccctcacagtggtggaactacctacaacgagaagttcaagggcaaggccacattgactgtaaataagtcctccagcacagccaacatggagctccgcagtctgacatcggaggattctgcagtctattactgtgcaagtatttattactacggtagtagctactttgaccactggggccaaggcaccactctcacagtctcctca

SEQ ID NO: 51 - protein, 120 aa, Mus musculus
  MISC_FEATURE: SC72.96 Heavy Chain Variable Region
EVQLQQSGPELVKPGASVKMSCKASGYTLTAYNIHWVKQSHGKSLEWIGFINPHSGGTTYNEKFKGKATLTVNKSSSTANMELRSLTSEDSAVYYCASIYYYGSSYFDHWGQGTTLTVSS

SEQ ID NO: 52 - DNA, 339 bp, Mus musculus
  misc_feature: SC72.132 Light Chain Variable Region
gacattgtgatgtcacagtctccatcctccctagctgtgtcagttggagagaaggttactctgagctgcaagtccagtcagacccttttatatagtagcaatcaacagaactacttggcctggtaccaacagaaaccagggcagtctcctaaactgctgatttactgggcatccactagggaatctggggtccctgatcgcttcacaggcagtggatctgggacagatttcacgctcaccatcagcagtgtgaaggctgaagacctggcagtttattactgtcaccaatattatacgtatccattcacgttcggctcggggacaaagttggaaataaaa

SEQ ID NO: 53 - protein, 113 aa, Mus musculus
  MISC_FEATURE: SC72.132 Light Chain Variable Region
DIVMSQSPSSLAVSVGEKVTLSCKSSQTLLYSSNQQNYLAWYQQKPGQSPKLLIYWASTRESGVPDRFTGSGSGTDFTLTISSVKAEDLAVYYCHQYYTYPFTFGSGTKLEIK

SEQ ID NO: 54 - DNA, 360 bp, Mus musculus
  misc_feature: SC72.132 Heavy Chain Variable Region
gaagtgaagctggtggagtctgagggaggcttagtgcagcctggaagttccatgaaactctcctgcacagcctctggattcactttcagtgactattacatggcttgggtccgccaggttccagaaaagggtctagaatgggttgcaaccattgattatgatggtagtaacacctactatctggactccttgaagagccgtttcatcatctcgagagacaatgcaaagaacattctatacctgcaaatgagcagtctgaagtctgaggacacagccacgtattactgtgcaagagatggaggttacgacgaggactactttgactactggggccaaggcaccactctcacagtctcctca

SEQ ID NO: 55 - protein, 120 aa, Mus musculus
  MISC_FEATURE: SC72.132 Heavy Chain Variable Region
EVKLVESEGGLVQPGSSMKLSCTASGFTFSDYYMAWVRQVPEKGLEWVATIDYDGSNTYYLDSLKSRFIISRDNAKNILYLQMSSLKSEDTATYYCARDGGYDEDYFDYWGQGTTLTVSS

SEQ ID NO: 56 - DNA, 333 bp, Mus musculus
  misc_feature: SC72.165 Light Chain Variable Region
gacattgtgctgacccaatctccagcttctttggctgtgtctccagggcagagggccaccatctcctgcaaggccagccaaagtgttggttatgatggtgatacttatttgagctggtaccaacagaaaccaggacagccacccaaactcctcatctatggtgtatctattctagaatctgggatcccagccaggtttagtggcagtgggtctgggacagacttcaccctcaacatccatcctgtggaagaggaggatgctgcaacctattactgtcagcaaagtagtgaggatccgtacacgttcggaggggggaccaagttggagataaaa

SEQ ID NO: 57 - protein, 111 aa, Mus musculus
  MISC_FEATURE: SC72.165 Light Chain Variable Region
DIVLTQSPASLAVSPGQRATISCKASQSVGYDGDTYLSWYQQKPGQPPKLLIYGVSILESGIPARFSGSGSGTDFTLNIHPVEEEDAATYYCQQSSEDPYTFGGGTKLEIK

SEQ ID NO: 58 - DNA, 342 bp, Mus musculus
  misc_feature: SC72.165 Heavy Chain Variable Region
caggtccaactgcagcagcctggtgctgagcttgtgaggcctggggcctcagtgagactgtcctgcaaggcttctggctatagtttcaccagctactggataaactgggtgaagcagaggcctggacaaggccttgagtggattggaaatatttatcctggaagtagtaatactaactacaatgagaagttcaagaggaaggccacactgactgtagacacatcctccagcacagcctacatgcagctcagcagcctgacatctgacgactctgcggtctattattgtgcaagatcggagagtgactactggggccaaggcaccactctcacagtctcctca

SEQ ID NO: 59 - protein, 114 aa, Mus musculus
  MISC_FEATURE: SC72.165 Heavy Chain Variable Region
QVQLQQPGAELVRPGASVRLSCKASGYSFTSYWINWVKQRPGQGLEWIGNIYPGSSNTNYNEKFKRKATLTVDTSSSTAYMQLSSLTSDDSAVYYCARSESDYWGQGTTLTVSS

SEQ ID NO: 60 - DNA, 333 bp, Mus musculus
  misc_feature: SC72.181 Light Chain Variable Region
gacattgtgctgacccaatctccagcttctttggctgtgtctctagggcagagggccaccatctcctgcaaggccagccaaagtgttgatgatgatggtggtacttatatgagctggtaccaacagaaaccaggacagccacccaaactcctcatctatggtgtattcattctagaatctgggatcccagccaggtttagtggcagtgggtctgggacagacttcaccctcaacatccatcctgtggaagaggaggatgctgcaacctattactgtcagcaaagtaatgaggatccgtacacgttcggaggggggaccaagctggaaataaaa

SEQ ID NO: 61 - protein, 111 aa, Mus musculus
  MISC_FEATURE: SC72.181 Light Chain Variable Region
DIVLTQSPASLAVSLGQRATISCKASQSVDDDGGTYMSWYQQKPGQPPKLLIYGVFILESGIPARFSGSGSGTDFTLNIHPVEEEDAATYYCQQSNEDPYTFGGGTKLEIK

SEQ ID NO: 62 - DNA, 342 bp, Mus musculus
  misc_feature: SC72.181 Heavy Chain Variable Region
caggtccaactgcagcagcctggtgctgaggttgtgaagcctggggcctcagtgagactgtcctgcaaggcttctggctacactttcaccagctactggataaactgggtgaaacagaggcctggacaaggccttgagtggattggtaatatttatcctggtagtagtagtactaactacaatgagaagttcaagagcaaggccacactgactgtagacacatcctccagcacagcctacatgcagctcagcagcctgacatctgacgactctgcggtctattattgttcaagatcggagagtgactactggggccaaggcaccactctcacagtctcctca

SEQ ID NO: 63 - protein, 114 aa, Mus musculus
  MISC_FEATURE: SC72.181 Heavy Chain Variable Region
QVQLQQPGAEVVKPGASVRLSCKASGYTFTSYWINWVKQRPGQGLEWIGNIYPGSSSTNYNEKFKSKATLTVDTSSSTAYMQLSSLTSDDSAVYYCSRSESDYWGQGTTLTVSS

SEQ ID NO: 64 - DNA, 321 bp, Mus musculus
  misc_feature: SC72.201 Light Chain Variable Region
gacatccagatgactcagtctccagcctccctatctgtatctgtgggagaaactgtcaccatcacatgtcgagcaagtgaaaatatttacagtagtttagcatggtatcagcagaaacagggaaattctcctcagctcctggtctatgctgcaacaaagttagcagatggtgtgccatcaaggttcagtggcagtggatcaggcacacagtattccctcaagatcaacagcctgcagtctgaagattttgggagttattactgtcaacatttttggggtactacgtggacgttcggtggaggcaccaagctggaaatcaaa

SEQ ID NO: 65 - protein, 107 aa, Mus musculus
  MISC_FEATURE: SC72.201 Light Chain Variable Region
DIQMTQSPASLSVSVGETVTITCRASENIYSSLAWYQQKQGNSPQLLVYAATKLADGVPSRFSGSGSGTQYSLKINSLQSEDFGSYYCQHFWGTTWTFGGGTKLEIK

SEQ ID NO: 66 - DNA, 345 bp, Mus musculus
  misc_feature: SC72.201 Heavy Chain Variable Region
gaagtgaaccttgaggagtctggaggaggcttggtgcaacctggaggatccatgaaactctcctgtgttgcctctggattcactttcagtaactactggatgaactgggtccgccagtctccagagaaggggcttgagtgggttgctcaaattagatcgaaatctgataattatgcaacacattatgcggagtctgtgaaagggaggttcaccatctcaagagatgattccaaaagtagtgtctacctgcaaatgaacaacttaaggtctgaagacactggaatttattactgcagaactggtcccgatgtctggggcacagggaccacggtcaccgtctcctca

SEQ ID NO: 67 - protein, 115 aa, Mus musculus
  MISC_FEATURE: SC72.201 Heavy Chain Variable Region
EVNLEESGGGLVQPGGSMKLSCVASGFTFSNYWMNWVRQSPEKGLEWVAQIRSKSDNYATHYAESVKGRFTISRDDSKSSVYLQMNNLRSEDTGIYYCRTGPDVWGTGTTVTVSS

SEQ ID NO: 68 - DNA, 333 bp, Mus musculus
  misc_feature: SC72.216 Light Chain Variable Region
gacattgtggtgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatggtgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaa

SEQ ID NO: 69 - protein, 111 aa, Mus musculus
  MISC_FEATURE: SC72.216 Light Chain Variable Region
DIVVTQSPASLAVSLGQRATISCKASQSVDYDGDSYMNWYQQKPGQPPKLLIYGASNLESGIPARFSGSGSGTDFTLNIHPVEEEDAATYYCQQSNEDPYTFGGGTKLEIK

SEQ ID NO: 70 - DNA, 360 bp, Mus musculus
  misc_feature: SC72.216 Heavy Chain Variable Region
cagatccagttggtgcagtctggacctgagctgaggaagcctggagagacagtcaagatctcctgcaaggcttctgggtatacgttcacaaactatccaatgcactgggtgaaacaggctccaggaaagggtttcaagtggatgggctggataaacacctactctggagtgccatcatatgcagatgacttcaagggacggtttgccttctctttggaaacctctgccagctctgcatatttgcagatcaacaacctcaaaaatgaggacatggctacatatttctgtgcaagacataattacaatagttacgacgtgtttgcttactggggccaagggactctggtcactgtctctgca

SEQ ID NO: 71 - protein, 120 aa, Mus musculus
  MISC_FEATURE: SC72.216 Heavy Chain Variable Region
QIQLVQSGPELRKPGETVKISCKASGYTFTNYPMHWVKQAPGKGFKWMGWINTYSGVPSYADDFKGRFAFSLETSASSAYLQINNLKNEDMATYFCARHNYNSYDVFAYWGQGTLVTVSA

SEQ ID NO: 72 - DNA, 342 bp, Mus musculus
  misc_feature: SC72.93 Heavy Chain Variable Region
caggttcaactgcagcagtctggggctgagctggtgaggcctggggcttcagtgacgctgtcctgcaaggcttcgggctacatatttattgactatgaaatgcactgggtgaagcagacacctgtgcatggcctggaatggattggatctattgatcctgagactggtgatactgtctacaatcagaagttcaagggcaaggccacactgactgcagacaaatcctccagcacagcctacatggagctccgcagcctgacatctgaggactctgccgtctattattgttcaagagggggtcttgactactggggccaaggcaccactctcacagtctcctca

SEQ ID NO: 73 - protein, 114 aa, Mus musculus
  MISC_FEATURE: SC72.93 Heavy Chain Variable Region
QVQLQQSGAELVRPGASVTLSCKASGYIFIDYEMHWVKQTPVHGLEWIGSIDPETGDTVYNQKFKGKATLTADKSSSTAYMELRSLTSEDSAVYYCSRGGLDYWGQGTTLTVSS